(19) United States
(12) Patent Application Publication
(10) Pub. No.: US 2025/0172577 A1
(43) Pub. Date: May 29, 2025

(54) METHODS FOR THE DETECTION AND TREATMENT OF CANCER

(71) Applicant: Board of Regents, The University of Texas System, Austin, TX (US)

(72) Inventors: Jennifer Bolin DENNISON, Houston, TX (US); Ranran WU, Houston, TX (US); Samir HANASH, Houston, TX (US); Marta SANS ESCOFET, Houston, TX (US); Anirban MAITRA, West University, TX (US); Johannes Francois FAHRMANN, Houston, TX (US); Eunice N. MURAGE, Missouri City, TX (US)

(21) Appl. No.: 18/969,577

(22) Filed: Dec. 5, 2024

Related U.S. Application Data
(63) Continuation of application No. PCT/US2023/067979, filed on Jun. 6, 2023.
(60) Provisional application No. 63/365,945, filed on Jun. 6, 2022.

(51) Int. Cl. G01N 33/92

(52) U.S. Cl. CPC G01N 33/92; G01N 2405/08; G01N 2560/00; G01N 2800/50

(57) ABSTRACT

Provided are methods and related kits for detection and treatment of early-stage pancreatic ductal adenocarcinoma. Also provided are methods for treating a patient susceptible, or suspected of being susceptible, to pancreatic ductal adenocarcinoma.

### CROSS-REFERENCE TO RELATED APPLICATIONS

This application is a bypass continuation of International Application No. PCT/US2023/067979, filed Jun. 6, 2023, which claims the benefit of priority of U.S. Provisional Application No. 63/365,945, filed Jun. 6, 2022, the contents of each are incorporated by reference as if written herein in their entireties.

### STATEMENT REGARDING FEDERALLY SPONSORED RESEARCH OR DEVELOPMENT

This invention was made with government support under grant number U01 CA200468, awarded by the National Institutes of Health. The government has certain rights in the invention.

### BACKGROUND

Pancreatic Ductal Adenocarcinoma (PDAC) is projected to become the second leading cause of cancer death by 2040. Around 2-3% of the general population harbor one or more pancreatic cysts, of which, intraductal papillary mucinous neoplasms (IPMNs) are the most common type of pancreatic cysts, which originate from normal ducts, acquiring higher grades of dysplasia. The majority of IPMNs are benign, although approximately 5-10% eventually culminate in PDAC. The 5-year survival rate for pancreatic ductal adenocarcinoma (PDAC), the third most common cause of cancer related deaths in the United States, is only 10%, with limited therapeutic options and a median survival of less than a year. As a result, the overwhelming majority (>80%) of patients present with locally advanced or distant metastatic disease, precluding surgical resection and the possibility of long-term cure. Nonetheless, the existence of asymptomatic precursor lesions—both microscopic (non-cystic) and macroscopic (cystic) in nature—that are known to predate invasive PDAC by years provides a compelling rationale for a “window of opportunity” for cancer interception efforts. Improved understanding of the molecular changes driving the malignant progression of IPMNs is needed to identify markers that can be translated for early detection strategies as well as towards new targetable features for therapeutic intervention. The present disclosure utilizes a unique multimodal approach combining spatial transcriptomics (ST) with matrix-assisted laser desorption ionization (MALDI) mass spectrometry (MS) to investigate gene expression and aberrant lipidomic changes correlated with higher grades of dysplasia and invasiveness in a cohort of resected IPMN samples.

### SUMMARY

Provided are methods for determining susceptibility of a patient having a pancreatic disorder characterized by elevated levels of long-chain hydroxylated sulfatide species progressing to pancreatic ductal adenocarcinoma (PDAC), comprising measuring the levels or amounts of long-chain hydroxylated sulfatide species in a biological sample obtained from the patient; wherein increased levels or amounts of long-chain hydroxylated sulfatide species in the patient classify the patient as being susceptible to developing pancreatic ductal adenocarcinoma (PDAC) from the pancreatic disorder.

Also provided are methods for treating or preventing progression of a pancreatic disorder to pancreatic ductal adenocarcinoma (PDAC) in a patient, comprising administering a therapeutically effective amount of an inhibitor of sulfatide metabolism to the patient.

Also provided are methods of treating a pancreatic disorder comprising: performing mass spectrometry imaging of sphingolipids to establish or delineate the borders of a pancreatic tumor or cyst; and surgically resecting the tumor or cyst.

These and other aspects of the invention will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1—Shows Kras;Gnas mice on Dox develop visible cystic lesions as early as 6 weeks post-induction (left, circle), which are confirmed as murine IPMNs on histopathology (center). Ultrasound confirms presence of cystic lesion in pancreatic tail (right).

FIG. 2—Shows representative MALDI-MS images of human IPMN. MALDI-MS images are provided for C24:1(OH) and C24:0(OH) sulfatides. IPMN areas are outlined in black on the H&E images. Outline of the tissue is drawn on the MALDI images in white.

FIG. 3—Shows detection of Sulfatide Species in Kras;Gnas Mice. (Top) MALDI-MS images showing distribution of C24:1(OH) and C24:0(OH) sulfatide in a Kras;Gnas mouse tissue section harvested after 25 weeks on doxycycline diet. H&E staining with higher magnification for two regions: (A) high grade dysplasia, (B) low grade dysplasia.

FIG. 4—Shows a schematic of the sulfatide metabolic pathway.

FIGS. 5A-5D—Shows spatial transcriptomics of human and murine IPMN. (FIG. 5A) Spatial maps showing expression of gene transcripts for enzymes involved in sulfatide metabolism for a LG IPMN sample (LG_2—same sample shown in FIG. 4 for MALDI-MS imaging). (FIG. 5B) Expression of UGT8 in a HG IPMN with adjacent diffuse PDAC. UGT8 is expressed in the IPMN and adjacent cancer areas but not in lymphocyte aggregates (yellow arrow). (FIG. 5C) UGT8 and FA2H levels detected by LCM followed by RNA sequencing from acinar (n=10), normal duct (n=16), and IPMN (n=40) samples. (FIG. 5D) Expression of Gal3st1 in cystic lesions collected from a Kras;Gnas mice harvested after 25 weeks on doxycycline diet. IPMN, islet, and lymphocyte aggregate areas are outlined in the H&E optical images in black, islet, and yellow, respectively.

FIG. 6—Shows Levels (log 2 area units) of sulfatide species quantified in cell lines established from Kras;Gnas mice.

FIGS. 7A-7C—Shows in vivo CRISPR KO tumor platform. (FIG. 7A) Schematic of the in vivo CRISPR KO platform. (FIG. 7B) Kaplan-Meier survival graph of CRISPR mice: KrasG12D (K), Trp53 KO (P), Lkb1 KO (L), Arid1a KO (A) and control (LacZ) mice. (FIG. 7C) Pancreas tumor immunofluorescence of Lkb1 (red) and DAPI (blue) in adeno-associated virus (AAV) KrasG12D; Trp53 KO (KP) and AAV KrasG12D; Trp53 KO; Lkb1 KO (KPL) mice.

FIGS. 8A-8F—Shows in vivo CRISPR activation platform. (FIG. 8A) Schematic of the CRISPR activation mouse model. (FIG. 8B) IF staining of Myc-activated pancreatic organoids (Cre/sgMyc) and non-targeted controls (Cre/sgNT). (FIG. 8C) qPCR analysis of Myc-activated organoids. (FIG. 8D) Retrograde ductal injection with blue tracer fluid in murine pancreas. (FIG. 8E) CRISPRa pancreas tumor, macroscopic image (top), mCherry fluorescence (bottom), (FIG. 8F) MYC (red), dCas9VP64 (green) and DAPI (blue) staining in Myc-activated (PPKS/M) and non-targeted tumor (PPKS/NT).

FIG. 9—Shows a distribution of sulfatide lipids correlate with PDAC. MALDI-MS images of cancer and normal-adjacent tissue from the same patient. Normal ducts, PDAC, and islet areas with pancreatitis are outlined in purple, black, and red, respectively, in the H&E images.

FIG. 10—Shows results of ST Analysis using a sample cohort of 13 samples (7 low-grade IPMN, 3 high-grade IPMN, and 3 PDAC samples).

FIG. 11—Shows region-specific analyses based on histology following ST Analysis using a sample cohort of 13 samples (7 low-grade IPMN, 3 high-grade IPMN, and 3 PDAC samples).

FIG. 12—Shows tumor immune microenvironment organization associated with IPMN grade and invasiveness. Data was obtained using single-cell data deconvolution by robust decomposition of cell type mixtures in spatial transcriptomics (RCTD) and scRNAseq FNA data.

FIG. 13—Shows a general overview of spatially resolved lipid analysis (ST analysis).

FIG. 14—Shows enrichment of cerebroside species in IPMN as obtained by MALDI-MS imaging,

FIG. 15—Shows long-chain hydroxylated sulfatides are elevated in PDAC.

FIG. 16—Shows the ratio of long to short chain sulfatide.

FIG. 17—Shows increased galactosylceramide synthesis in IPMN.

FIG. 18—Shows sulfatide acyl chain length and hydroxylation data.

FIG. 19—Shows elevation of arylsulfatase A in LG-IPMN.

FIG. 20—Shows colocalization of expression of genes of interest with sulfatide species.

FIG. 21—Shows sulfatide species detected in a murine model of IPMN.

FIG. 22—Shows sulfatide metabolism recapitulated in a murine model of IPMN.

FIGS. 23A-23B—(FIG. 23A) shows in vitro toxicity assays (MTS Assay) for three independent mouse IPMN cell lines (LGKC4301, LGKC4838, and LGKC4861) treated with vehicle control or the UGT8 inhibitors UGT8-IN-1 or zoledronic acid. Experiments were performed in technical triplicates. Statistical significance was determined by 2-sided Student T-test. (FIG. 23B) shows mouse IPMN Cell line LGKC4861 (p48-Cre; LSL-KrasG12D; Rosa26R-LSL-rtTA-TetO-GnasR201C) were implanted in 8-week-old male and female athymic nude mice subcutaneously (1×10⁵ cells per mouse) with doxycycline (200 μg/ml) supplemented in drinking water. UGT8 inhibitor UGT8-IN-1 or vehicle control (saline) was orally administrated every other day (3 mg/kg) from day-8 post cancer cell implantation for 2 weeks. Tumor volume was measured every 3 days. Statistical significance was determined by 2-way repeated measures ANOVA and treatment factor 2-sided p-value reported.

### DETAILED DESCRIPTION

### Overview

The present disclosure describes a multi-omics approach, utilizing (1) clinical specimens, from patients undergoing IPMN surgery, in addition to tissue samples from a Kras;Gnas model that recapitulates the development of IPMN in humans; (2) fresh frozen samples for spatial transcriptomics to look at gene expression profiles and localization in IPMN and PDAC, and then on serial sections performed MALDI-MS imaging for lipidomic analyses; (3) another cohort of fixed tissues was also used for LCM+RNAseq; and (4) cyst fluid samples for LC-MS/MS analyses. Using MALDI-MS metabolite imaging of tissue sections from resected IPMN cases and from a genetically engineered mouse model of IPMN, the present disclosure identifies novel discrete lipid signatures that are manifest at the earliest presentation of pre-neoplastic lesions and persists through progression to carcinoma. The biological role(s) of sulfatide in this context has, to-date, been unexplored, especially in the context of early preneoplasia. The goals of the present study included: (1) definition of the biological role(s) of sulfatide in the development and progression of pancreatic cancer precursor lesions; (2) testing whether small molecule inhibition of sulfatide metabolism is a viable cancer interception strategy; and (3) establishing the potential clinical utility of imaging-based assessment of sulfatides for identifying intraoperative margins and metastatic nodules from pancreatic surgeries.

The present disclosure additionally provides (1) the use of patient-derived organoids from pancreatic precursor lesions, as well as genetically engineered mouse models that recapitulate pancreatic cystic neoplasia, which can be used to interrogate the functional relevance of sulfatide; (2) organ-site specific CRISPR-based knockout and activation techniques to genetically manipulate key enzymes involved in sulfatide metabolism in vivo; and (3) integrated mass spectrometry-based proteomics, lipidomics, stable-isotope resolved metabolomics, and transcriptomic profiling analyses to establish molecular changes and identify key signaling pathways.

In some embodiments, the present disclosure provides methods of treating or preventing a cancer, e.g., PDAC, by targeting synthesis of a sulfatide species. In some embodiments, treatment or prevention of a cancer may be accomplished by targeting an enzyme that is involved in synthesis of a sulfatide species, such as an enzyme disclosed herein. Targeting expression may be accomplished by any method known or available in the art, e.g., miRNA, RNA interference, or the like.

### Increasing Prevalence of Pancreatic Cysts

Pancreatic cysts occur in 2.4% to 13% of patients studied by abdominal imaging (CT scan or MRI) for reasons unrelated to pancreatic symptoms. In light of the ever-increasing use of abdominal imaging, the prevalence of incidental detection of pancreatic cysts continues to rise, with almost half a million new pancreatic cysts diagnosed each year in the United States alone, particularly in the older population. Retrospective histopathology studies on surgically resected pancreatic cysts have shown that approximately half are neoplastic entities. The most common cystic neoplasm that is a bona fide precursor to pancreatic ductal adenocarcinoma (PDAC) is intraductal papillary mucinous neoplasm (IPMN). As the name suggests, IPMN is a mucin-secreting neoplasm that arises in either the main pancreatic duct or one of the branch ducts. IPMNs comprise roughly 40-50% of resected lesions that are initially diagnosed as asymptomatic pancreatic cysts. IPMNs are lined by either low-grade (LG) or high-grade (HG) epithelial dysplasia, and in a subset of cases, histological progression culminates in cancer. Once an IPMN develops an invasive component, the probability of long-term survival drops, reiterating the need for early detection and interception prior to the development of an invasive cancer. Evolutionary modeling studies suggest that for most patients with IPMNs, there is a significant window of time (at least 3-7 years) between non-invasive IPMNs and subsequent PDAC, providing an opportunity for surveillance and intervention. Currently, surgical resection is the only available definitive treatment modality for IPMNs, highlighting the importance of identifying molecular vulnerabilities that would substantially delay or eliminate the progression of these pancreatic early lesions to invasive PDAC.

### Sulfatide Compounds

Sulfatide compounds useful as described herein may include, but are not limited to, the following.

| Sulfatide | | |
|---|---|---|
| Compound | Synonym | IUPAC Name |
| C24:1(OH) | (3′-sulfo)Galbeta- | [(2R,5S,6R)-3,5-dihydroxy-2-[(E,2S,3R)-3- |
| Sulfatide | Cer(d18:0/2-OH- | hydroxy-2-[[(Z)-2-hydroxytetracos-15- |
| | 24:1(15Z)) | enoyl]amino]octadec-4-enoxy]-6- |
| | | (hydroxymethyl)oxan-4-yl] hydrogen sulfate |
| C24:0(OH) | (3′-sulfo)Galbeta- | N-(2-hydroxy-tetracosanoyl)-1-β-(3′-sulfo)- |
| Sulfatide | Cer(d18:1/24:0(2OH)) | glucosyl-sphing-4-enine |
| C24:0 | (3′-sulfo)Galbeta- | [(2R,3S,6R)-3,5-dihydroxy-2-(hydroxymethyl)-6- |
| Sulfatide | Cer(d18:1/24:0) | [(E,2S,3R)-3-hydroxy-2- |
| | | (tetracosanoylamino)octadec-4-enoxy]oxan-4-yl] |
| | | hydrogen sulfate |
| C16:0 | (3′-sulfo)Galbeta- | [(2R,3R,4S,5S,6R)-2-[(E,2S,3R)-2- |
| Sulfatide | Cer(d18:1/16:0) | (hexadecanoylamino)-3-hydroxyoctadec-4- |
| | | enoxy]-3,5-dihydroxy-6-(hydroxymethyl)oxan-4- |
| | | yl] hydrogen sulfate |
| C18:0 | (3′-sulfo)GalBeta- | [(2R,3S,6R)-3,5-dihydroxy-2-(hydroxymethyl)-6- |
| Sulfatide | Cer(d18:1/18:0) | [(E,2S,3R)-3-hydroxy-2- |
| | | (octadecanoylamino)octadec-4-enoxy]oxan-4-yl] |
| | | hydrogen sulfate |
| C20:0 | (3′-sulfo)Galbeta- | [(2R,5S,6R)-3,5-dihydroxy-2-[(E,2S,3R)-3- |
| Sulfatide | Cer(d18:1/20:0) | hydroxy-2-(icosanoylamino)octadec-4-enoxy]-6- |
| | | (hydroxymethyl)oxan-4-yl] hydrogen sulfate |
| C22:0 | (3′-sulfo)Galbeta- | [(2R,5S,6R)-2-[(E,2S,3R)-2-(docosanoylamino)-3- |
| Sulfatide | Cer(d18:1/22:0) | hydroxyoctadec-4-enoxy]-3,5-dihydroxy-6- |
| | | (hydroxymethyl)oxan-4-yl] hydrogen sulfate |

### Diagnosis, Staging, and Treatment of Pancreatic Cancer.

The most common way to classify pancreatic cancer is to divide it into 4 categories based on whether it can be removed with surgery and where it has spread: resectable, borderline resectable, locally advanced, or metastatic. Resectable pancreatic cancer can be surgically removed. The tumor may be located only in the pancreas or extends beyond it, but it has not grown into important arteries or veins in the area. There is no evidence that the tumor has spread to areas outside of the pancreas. Using standard methods common in the medical industry today, only about 10% to 15% of patients are diagnosed with this stage. Borderline resectable describes a tumor that may be difficult, or not possible, to remove surgically when it is first diagnosed, but if chemotherapy and/or radiation therapy is able to shrink the tumor first, it may be able to be removed later with negative margins. A negative margin means that no visible cancer cells are left behind. Locally advanced pancreatic cancer is still located only in the area around the pancreas, but it cannot be surgically removed because it has grown into nearby arteries or veins or to nearby organs. However, there are no signs that it has spread to any distant parts of the body. Using standard methods common in the medical industry today, approximately 35% to 40% of patients are diagnosed with this stage. Metastatic means the cancer has spread beyond the area of the pancreas and to other organs, such as the liver or distant areas of the abdomen. Using standard methods common in the medical industry today, approximately 45% to 55% of patients are diagnosed with this stage. Alternatively, the TNM Staging System, commonly used for other cancers, may be used (but is not common in pancreatic cancer). This system is based on tumor size (T), spread to lymph nodes (N), and metastasis (M).

Options for treatment of pancreatic cancer include surgery for partial or complete surgical removal of cancerous tissue (for example a Whipple procedure, distal pancreatectomy, or total pancreatectomy), administering one or more chemotherapeutic drugs, and administering therapeutic radiation to the affected tissue (e.g., conventional/standard fraction radiation therapy stereotactic body radiation (SBRT)). Chemotherapeutic drugs approved for treatment of pancreatic cancer include, but are not limited to, capecitabine (Xeloda), erlotinib (Tarceva), fluorouracil (5-FU), gemcitabine (Gemzar), irinotecan (Camptosar), leucovorin (Wellcovorin), nab-paclitaxel (Abraxane), nanoliposomal irinotecan (Onivyde), and oxaliplatin (Eloxatin).

Pancreatic cancer is treated most effectively when diagnosed early, preferably at or before the borderline resectable stage and more preferably at the resectable stage.

In some embodiments, the present methods and disclosure may be useful for treatment or prevention of progression of any cancer that can be classified as having elevated levels or amounts of a cerebroside species and/or a sulfatide species, such as including, but not limited to, pancreatic cancer, e.g., pancreatic ductal adenocarcinoma, or breast cancer. In addition, non-cancerous disorders or conditions may also benefit from the present methods, such as intraductal papillary mucinous neoplasm (IPMN) or pancreatic cysts, as described herein.

In some embodiments, the disclosure provides imaging methods for pancreatic cysts or cancer types comprising the use of mass spectrometry to measure sulfatide or cerebroside species for imaging of pancreatic lesions and cancer. Sphingolipid species have been previously characterized by mass spectrometry imaging using normal murine and human pancreas, but have not been evaluated in cancer. Mass spectrometry approaches have also been proposed for surgical margin evaluation in pancreatic cancer resections, the use of sulfatide profiling in this context has not been explored. Detection of sphingolipid species, e.g., cerebroside and sulfatide species, may in some embodiments be useful for assessing the margins of a cyst or tumor, providing a benefit for surgical resection.

### Definitions

As used herein, the term “pancreatic cancer” means a malignant neoplasm of the pancreas characterized by the abnormal proliferation of cells, the growth of which cells exceeds and is uncoordinated with that of the normal tissues around it.

As used herein, the term “PDAC” refers to pancreatic ductal adenocarcinoma, which is pancreatic cancer that can originate in the ducts of the pancreas.

As used herein, the term “PDAC-positive” refers to classification of a subject as having PDAC.

As used herein, the term “PDAC-negative” refers to classification of a subject as not having PDAC.

As used herein, the term “pancreatitis” refers to an inflammation of the pancreas. Pancreatitis is not generally classified as a cancer, although it may advance to pancreatic cancer.

As used herein, the term “subject” or “patient” as used herein refers to a mammal, preferably a human, for whom a classification as PDAC-positive or PDAC-negative is desired, and for whom further treatment can be provided.

As used herein, a “reference patient” or “reference group” refers to a group of patients or subjects to which a test sample from a patient suspected of having or being susceptible to PDAC may be compared. In some embodiments, such a comparison may be used to determine whether the test subject has PDAC. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual having a healthy pancreas, or normal, non-compromised pancreatic function. A healthy patient or subject has no symptoms of PDAC or other pancreatic disease. In some embodiments, a healthy patient or subject may be used as a reference patient for comparison to diseased or suspected diseased samples for determination of PDAC in a patient or a group of patients.

As used herein, a “long-chain sulfatide species” or a “very long-chain sulfatide species” refers to a sulfatide having a number of carbon atoms in a chain of 13 or more, or from about 13 to about 24, including 13, 14, 15, 16, 17, 18, 19, 20, 21, 22, 23, or 24 carbon atoms, or the like. As used herein, a “long-chain hydroxylated sulfatide species” or a “very long-chain hydroxylated sulfatide species” refers to a sulfatide species having greater than 13 carbon atoms, or from about 13 to about 24 carbon atoms and further having a hydroxyl group attached. A very long-chain hydroxylated sulfatide species may also refer to a sulfatide species having greater than 22 carbon atoms, e.g., 22, 23, 24, 25, or 26 carbon atoms, or the like.

As used herein, a “cerebroside species” refers to a class of glycosphingolipids having a single carbohydrate, called monoglycosylceramides, which are important components in animal muscle and nerve cell membranes. Cerebroside species consist of a ceramide with a single sugar residue (glucose or galactose) at the 1-hydroxyl moiety. Cerebroside species with a glucose sugar are also referred to as glucocerebrosides or glucosylceramides, while cerebroside species with a galactose sugar are also referred to as galactocerebrosides or galactosylceramides. Cerebroside species useful in accordance with the present disclosure may include any cerebrosides known in the art, e.g., including, but not limited to, β-D-Galactosylceramide and 0-D-Glucosylceramide.

As used herein, a “sulfatide species” refers to a class of sulfoglycolipids, which contain a sulfate group. Sulfatides are a sub-type of cerebroside. Sulfatide species are synthesized primarily starting in the endoplasmic reticulum and ending in the Golgi apparatus, where ceramide is converted to galactocerebroside and later sulfated to make sulfatide. Sulfatide species are found primarily in the plasma membrane of oligodendrocytes of the central nervous system and also in the Schwann cells of the peripheral nervous system. Sulfatide species useful in accordance with the present disclosure include both non-hydroxylated and hydroxylated forms. Sulfatide species can include, but are not limited to, C24(OH) sulfatide, C24:0(OH) sulfatide, C24:1(OH) sulfatide, C16 sulfatide, C16(OH) sulfatide, C18 sulfatide, C18:0(OH) sulfatide, C20 sulfatide, C20:0(OH) sulfatide, C22 sulfatide, C22:1 sulfatide, C22:1(OH) sulfatide, C24 sulfatide, C24:1 sulfatide, C26 sulfatide, and C26:1 sulfatide.

Sulfatides are a sub-type of cerebroside that have a singular carbohydrate, and also an additional sulfate group.

As used herein, “high levels” or “elevated levels” or “elevated amounts” refers to an increase in the levels or amounts of a cerebroside species or a sulfatide species relative to healthy pancreatic tissue. Healthy pancreatic tissue may be pancreatic tissue from the same patient or individual, or may be from a healthy control.

As used herein, “UGT8,” or “ceramide galactosyltransferase,” or “CGT” all refer to the enzyme that catalyzes the addition of a galactose sugar group to ceramide to produce C26:0(OH), galactosylceramide in the endoplasmic reticulum (ER).

Ceramides can either get added to a compound as a glucose or a galactose. With glucose, UDP-glucose ceramide glucosyltransferase (UGCG) is the enzyme responsible, which is typically further modified into LacCer, a building block for larger chain sphingolipids, such as gangliosides. On the other hand, the addition of a galactose is catalyzed by UGT8, which can then have a sulfate group added, resulting in a sulfatide.

As used herein, a “ceramide” is defined by having a C18:1 sphingosine backbone, with the other fatty acyl group being variable. For example, a C16 ceramide refers to a ceramide having a C18:1 sphingosine backbone and a C16 group (18:1/16:0).

As used herein, a “sulfatide metabolizing enzyme” refers to an enzyme involved in the synthesis or production of a sulfatide species. An enzyme involved in the synthesis or production of a sulfatide species may include, but is not limited to, ceramide galactosyltransferase (UGT8); galactose-3-O-sulfotransferase (Gal3St1); arylsulfatase a (ARSA); ceramide synthase 2 (CERS2), or fatty-acid 2-hydroxylase (FA2H).

Sulfatide metabolizing enzymes can either be overexpressed (activated) or knocked down/knocked out (suppressed) in the pancreas. UGT8 is responsible for making galactosylceramides, which subsequently undergo sulfation via Gal3St1 to yield a sulfatide derivative.

As used herein, “Gal3st1,” or “galactose-3-O-sulfotransferase,” or “CST” all refer to the enzyme that catalyzes the addition of a sulfate group to the galactose residue on galactosylceramide in the Golgi apparatus.

As used herein, a “biological sample” refers to any sample obtained from a patient as described herein. A biological sample may be any type of sample useful for measurement of a cerebroside species or a sulfatide species, such as including, but not limited to, blood, serum, pancreatic tissue, or the like. A biological sample may be used in accordance with any of the methods described herein, such as including, but not limited to, measurement of levels or amounts of a species described herein, tissue sectioning, microarray analysis, tissue staining (e.g., hematoxylin/eosin), proteomics analysis, nucleic acid (e.g., DNA, RNA) analysis, gene expression studies, e.g., CRISPR,

As used herein, an “effective amount” of a compound, drug, or other agent refers to an amount that is sufficient to generate a desired response, such as reduce or eliminate a sign or symptom of a condition or disease. For instance, as described herein, an effective amount may be an amount necessary to prevent or treat a pancreatic disease, such as a pancreatic cyst or pancreatic cancer. When administered to a patient, a dosage will generally be used that will achieve target tissue concentrations (for example, in pancreatic tissue or surrounding areas) that have been shown to inhibit or prevent pancreatic disease or cancer. In some examples, an “effective amount” is one that treats (including prophylaxis) one or more symptoms and/or underlying causes of a disorder or disease. In some examples, an “effective amount” is one that inhibits the production of sulfatides or cerebrosides, or intermediates in a pathway involved in the production of these compounds, such that the levels of sulfatides or cerebrosides are reduced in a patient. In one example, an effective amount is a therapeutically effective amount. In one example, an effective amount is an amount that prevents one or more signs or symptoms of a particular disease or condition from developing.

The term “treatment” or “treating” as used herein refers to the administration of medicine or the performance of medical procedures with respect to a subject, for either prophylaxis (prevention) or to cure or reduce the extent of or likelihood of occurrence or recurrence of the infirmity or malady or condition or event in the instance where the subject or patient is afflicted. As related to the present disclosure, the term may also mean the administration of pharmacological substances or formulations, or the performance of non-pharmacological methods including, but not limited to, radiation therapy and surgery. Pharmacological substances as used herein may include, but are not limited to, chemotherapeutics that are established in the art, such as Gemcitabine (Gemzar), 5-fluorouracil (5-FU), irinotecan (Camptosar), oxaliplatin (Eloxatin), albumin-bound paclitaxel (Abraxane), capecitabine (Xeloda), cisplatin, paclitaxel (Taxol), docetaxel (Taxotere), and irinotecan liposome (Onivyde). Pharmacological substances may include substances used in immunotherapy, such as checkpoint inhibitors. Treatment may include a multiplicity of pharmacological substances, or a multiplicity of treatment methods, including, but not limited to, surgery and chemotherapy.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for PDAC-positive.

As used herein, the term “logistic regression” refers to a regression method in which the assignment of a prediction from the model can have one of several allowed discrete values. For example, the logistic regression models used herein can assign a prediction, for a certain subject, of either PDAC-positive or PDAC-negative.

As used herein, the term “biomarker score” refers to a numerical score for a particular subject that is calculated by inputting the particular biomarker levels for said subject to a statistical method.

As used herein, the term “cutoff point” refers to a mathematical value associated with a specific statistical method that can be used to assign a classification of PDAC-positive of PDAC-negative to a subject, based on said subject's biomarker score.

As used herein, the term “classification” refers to the assignment of a subject as either PDAC-positive or PDAC-negative, based on the result of the biomarker score that is obtained for said subject.

As used herein, the term “PDAC-positive” refers to an indication that a subject is predicted as susceptible to PDAC, based on the results of the outcome of the methods of the disclosure.

As used herein, the term “PDAC-negative” refers to an indication that a subject is predicted as not susceptible to PDAC, based on the results of the outcome of the methods of the disclosure.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or presence of PDAC in subjects of a certain population.

As used herein, the term “true positive rate” refers to the probability that a given subject classified as positive by a certain method is truly positive.

As used herein, the term “false positive rate” refers to the probability that a given subject classified as positive by a certain method is truly negative.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for positive-PDAC and non-positive-PDAC subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, the term “ctDNA” refers to cell-free or circulating tumor DNA. ctDNA is tumor DNA found circulating freely in the blood of a cancer patient. Without being limited by theory, ctDNA is thought to originate from dying tumor cells and can be present in a wide range of cancers but at varying levels and mutant allele fractions. Generally, ctDNA carry unique somatic mutations formed in the originating tumor cell and not found in the host's healthy cells. As such, the ctDNA somatic mutations can act as cancer-specific biomarkers.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, acetylspermidine, diacetylspermine, lysophosphatidylcholine (18:0), lysophosphatidylcholine (20:3) and an indole-derivative.

The foregoing has outlined rather broadly the features and technical benefits of the disclosure in order that the detailed description may be better understood. It should be appreciated by those skilled in the art that the specific embodiments disclosed may be readily utilized as a basis for modifying or designing other structures or processes for carrying out the same purposes of the disclosure. It is to be understood that the present disclosure is not limited to the particular embodiments described, as variations of the particular embodiments may be made and still fall within the scope of the appended claims.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of ordinary skill in the art should, in light of the present disclosure, appreciate that many changes can be made in the specific embodiments which are disclosed and still obtain a like or similar result without departing from the spirit and scope of the disclosure.

### Example 1—a Genetically Engineered Mouse Model Recapitulating Pancreatic Cystic Neoplasia

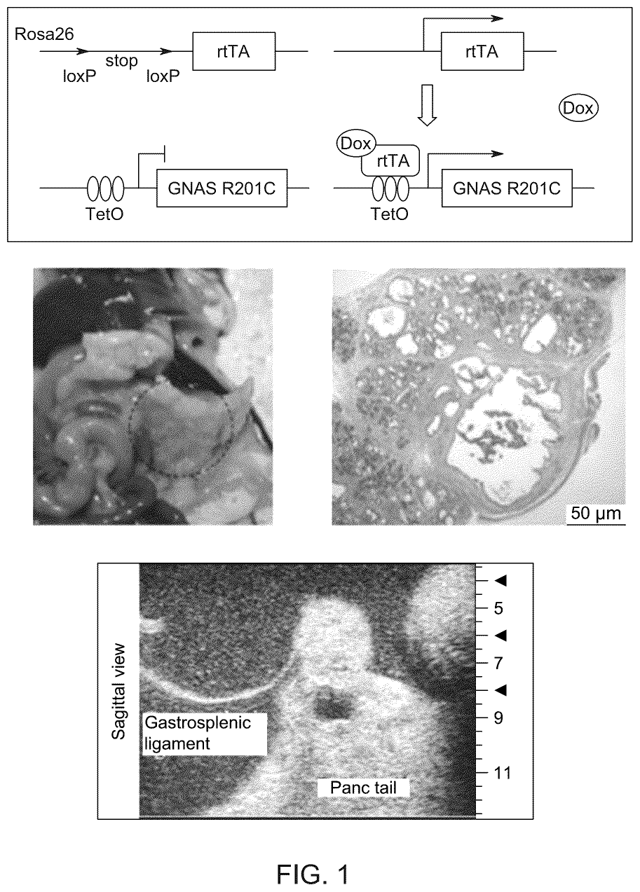

In 2012, the comprehensive genomic landscape of non-invasive IPMNs was elucidated for the first time. Point mutations in the KRAS oncogene at codons 12 or 13, considered the defining initiating mutations in non-cystic precursor lesions (i.e., pancreatic intraepithelial neoplasia or PanIN), are seen in ˜80% of IPMNs. However, IPMNs also contain somatic mutations in genes that are not frequently altered in PanINs, highlighting the unique molecular pathogenesis of the cystic pathway of pancreatic neoplasia. One example is “hotspot” activating mutations of GNAS, which encodes for the alpha subunit of the G-protein. Previous studies have shown that GNAS mutations are rather uncommon in non-cystic precursor (PanIN) lesions. To model the co-expression of mutant Kras and Gnas in the murine pancreatic epithelium, a doxycycline (Dox)-inducible GNAS^{R201C} model was crossed into the pancreas-specific Kras mutant background (Ptf1a^{Cre}; Kras^{LSL-G12D}; R26^{LSL-rtTA}, Tg(GNAS-R201C)) mice, henceforth referred to as Kras;Gnas mice for brevity) (FIG. 1). Compound heterozygotes were generated—in this model, mutant Kras expression is restricted to the pancreas, while co-expression of mutant Gnas is regulated by addition of Dox to drinking water. In the absence of Dox (“Dox Off”), i.e., with mutant Kras expression alone, the pancreata demonstrate murine pancreatic intraepithelial neoplasms (or PanINs), which are non-cystic precursor lesions of PDAC. However, upon co-expression of mutant Gnas (“Dox On”), ˜100% of the mice develop cystic lesions beginning as early as 6 weeks of doxycycline, readily visible by abdominal ultrasound (FIG. 1). In approximately 25% of Kras;Gnas mice, progression to invasive PDAC was observed that occurs on backdrop of high-grade dysplasia in IPMNs. The median onset of PDAC occurs at 21 weeks post Dox induction, which provides a “window of opportunity” for cancer interception, in the setting of non-invasive IPMNs. Multiple isogenic cell lines have been generated from PDACs arising in this model, which will be pivotal for the proposed studies.

### Example 2—Cross-Species MALDI-MS Imaging Reveals Long-Chain Hydroxylated Sulfatide to be Highly Specific to the Neoplastic Epithelium in IPMNs

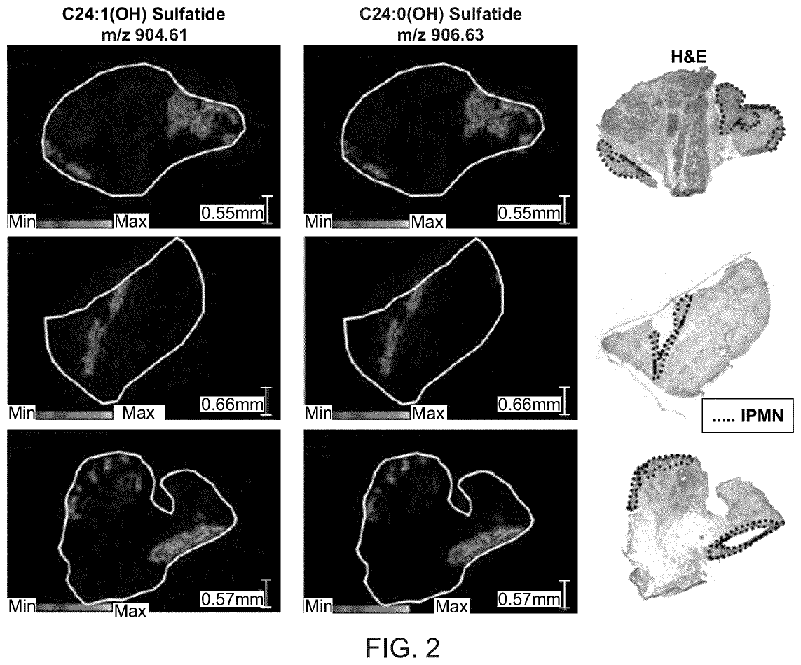

Matrix-assisted laser desorption/ionization (MALDI)-mass spectrometry (MS) was applied to perform an unbiased spatial characterization of lipid profiles in frozen tissue sections from resected human IPMN cases. MALDI-MS imaging revealed the detection of various lipid species, including fatty acids, lysophospholipids, glycerophospholipids, and sphingolipids, among others, in the tissue sections. Remarkably, MALDI analyses revealed cerebroside and sulfatide species, C24:1(OH) sulfatide (m/z 904.61), and C24:0(OH) sulfatide (m/z 906.63), among others, to be highly enriched and specific to areas outlining the IPMN epithelium. Importantly, these sulfatide species, particularly C24:1(OH) and C24:0(OH), were not detected by MALDI-MS in the surrounding stroma or normal pancreas, including acinar and islet cells, as shown in FIG. 2.

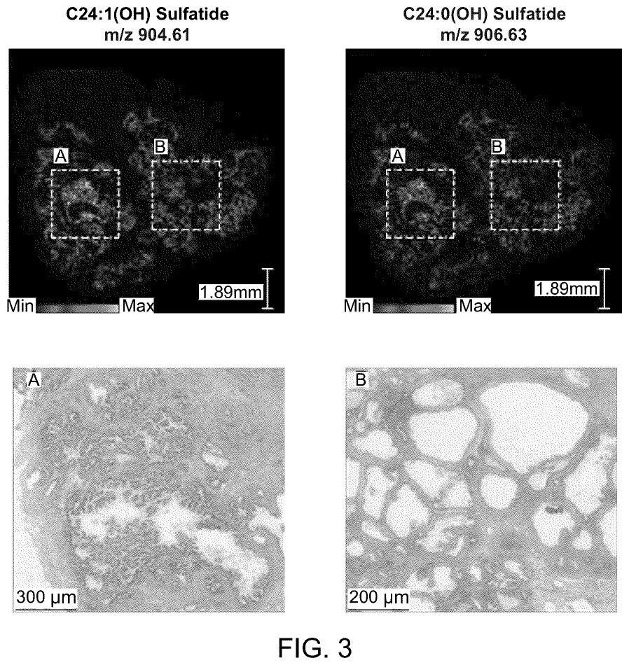

Sulfatide species were also detected by MALDI-MS imaging in tissue sections from resected cystic lesions from Kras;Gnas mice, which recapitulate the neoplastic progression of IPMN observed in humans. Both the C24:1(OH) and C24:0(OH) sulfatide were identified in the tissue samples, showing a similar spatial distribution and outlining the epithelial cystic lining, as shown in FIG. 3 for a neoplasm collected from a Kras;Gnas mice after 25 weeks on doxycycline diet. A higher relative abundance of the C24:1(OH) and C24:0(OH) sulfatide was also observed in the cystic areas presenting with a higher grade of dysplasia (FIG. 3A) compared to the surrounding smaller cysts with lower grade of dysplasia (FIG. 3B).

### Example 3—Spatial Transcriptomics of IPMNs Reveals Elevated Expression of Transcripts for Sulfatide Metabolizing Enzymes, Including UGT8 and Gal3ST1

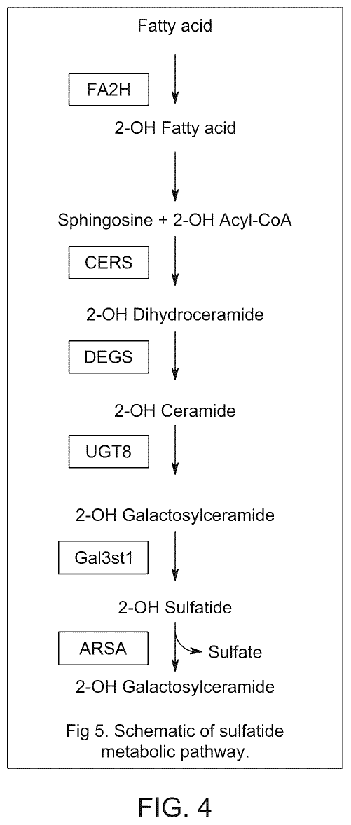

Sulfatides are a subtype of sphingolipid typically found in myelin sheath. Sulfatides are variable in structure, containing different lengths of acyl chain and ceramide moiety, which can be hydroxylated, as well as other sphingolipids. Sulfatide synthesis begins in the endoplasmic reticulum by the addition of galactose from UDP-galactose to ceramide, which is catalyzed by the ceramide galactosyltransferase (CGT, also known as UGT8) followed by O-sulfation of the galactose residue on galactosylceramide via galactose-3-O-sulfotransferase (CST, also known as Gal3St1) that occurs in the Golgi apparatus. The 3-O-sulfate group of sulfatide is hydrolyzed by arylsulfatase a (ASA; also referred to as ARSA). FIG. 4 shows a schematic of the sulfatide metabolic pathway.

Utilizing serial sections to the same human IPMN cases analyzed with MALDI-MS imaging, the Visium Spatial Gene Expression technology platform (10× Genomics, CA) was used to characterize the spatio-molecular underpinnings of LG and HG IPMN, including samples with co-occurring PDAC.

Spatial Transcriptomics technology (ST technology) is a way to correlate gene expression and tissue localization. Briefly, for ST experiments, a tissue specimen is sectioned (˜10 μm sections), stained, and imaged under a microscope. Tissue permeabilization and library construction is done, followed by sequencing, analysis, and visualization. This results in a spatial gene expression slide, which has been coded with capture areas that will capture tissue mRNA after permeabilization. Each “spot” is approximately 55 μm. The capture oligonucleotides are barcoded to match the mRNA to each location.

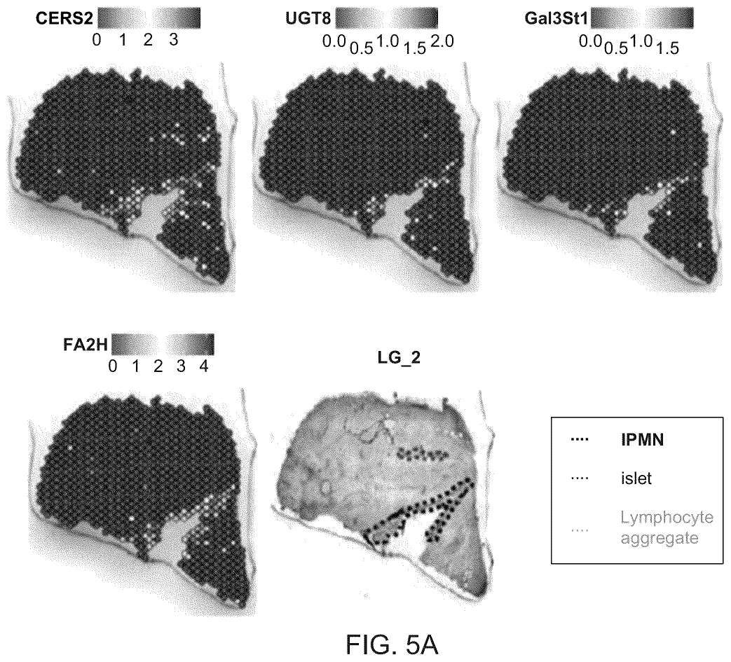

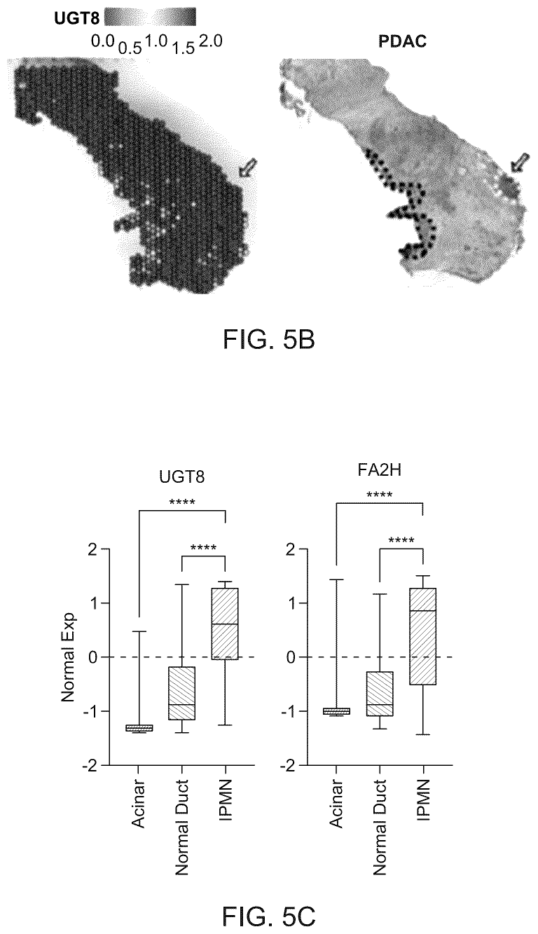

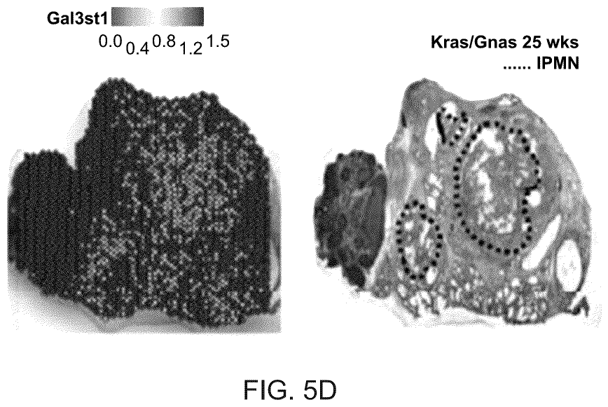

H&E images of the same tissue section used for the Visium workflow, were used to annotate the spots in the dataset covering the epithelial lining of the IPMN lesions (“epilesional” areas), as well as the spots covering the adjacent microenvironment surrounding the lesion (‘perilesional” areas). This approach enables generation of regional-specific gene expression profiles associated with grades of dysplasia in IPMN. Several transcripts encoding enzymes involved in sulfatide metabolism were identified to be elevated in the epilesional compartments of the IPMN and associated PDAC samples (FIG. 5A). CERS2 transcripts, encoding a ceramide synthase with high affinity for incorporating longer chain fatty acids (C22-C24), were detected in about 60% of all spots covering the epithelial compartments of IPMN and PDAC samples. Transcripts for UGT8 as well as GAL3ST1 were also found to be detected predominantly in the epilesional compartments, with a 3.3- and 3.6-fold increase, respectively, in number of spots expressing these transcripts in the epilesional versus all other areas in the tissue samples. These findings suggest an enrichment of the galactose-, and sulfatide-, related sphingolipid pathway in neoplastic epithelial cells compared to the adjacent stromal and immune microenvironment, consistent with the MALDI-MS imaging findings (FIG. 5B). Notably, Fatty-Acid 2-Hydroxylase (FA2H), which hydroxylates N-acyl chains during de novo ceramide synthesis (FIG. 4), was also found to be broadly expressed in the epilesional areas from IPMN and PDAC tissue samples. Importantly, UGT8 and FA2H transcripts were found to be statistically significantly elevated in IPMN compared to normal duct epithelium and acinar cells, using liquid capture microdissection coupled with RNA sequencing on an orthogonal cohort of fixed pancreatic tissue sections, suggesting an upregulation of cerebroside and hydroxylation in IPMN (FIG. 5C). Spatial transcriptomics analysis with the Visium technology on pancreatic tissue samples collected from Kras;Gnas mice after 25 weeks on doxycycline diet also yielded detectable transcripts for sulfatide-related enzyme transcripts including Cers2, Ugt8a, Gal3st1, Fa2h, and Arsa. As shown in FIG. 5D, high levels of Gal3st1 were observed in the cystic lesion areas. Taken together, these results coupled with the detection of sulfatide species by MALDI-MS imaging further supports sulfatide as a metabolic feature that is associated with IPMN development.

### Example 4—MALDI-MS Imaging of Sulfatide Species in Pancreatic Cancer Resections

Surgery remains the only current curative option for patients with resectable pancreatic cancer. To guide surgical resection and ensure adequate removal of the cancer, tissue margins from the pancreatic neck, uncinate process and common bile duct, among others, are commonly assessed by intraoperative frozen section evaluation. However, detection of pancreatic cancer at these margins for intraoperative assessment may be missed with currently used routine frozen section techniques, leading to misguidance on additional surgical need, which leads to an even poorer prognosis for the patient. Consequently, there is a pressing need to incorporate molecular information to improve accuracy of pancreatic cancer margin evaluation and help guide pancreatic cancer resection.

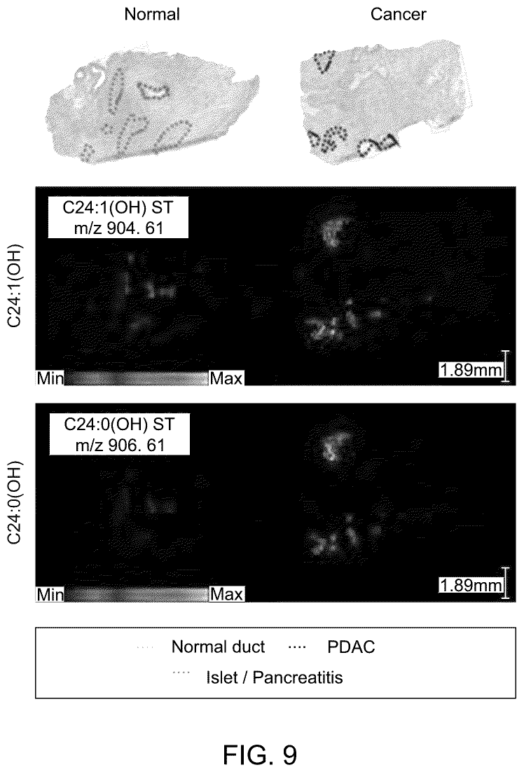

Preliminary studies showed high relative abundance of sulfatide species, particularly C24:1(OH) and C24:0(OH), in the epithelial linings of human and murine IPMN tissue samples (FIGS. 2-3). Sulfatide lipid species were also identified in PDAC tissue areas, both from cases with and without co-occurring IPMN, suggesting that sulfatide lipids are present in neoplastic ducts at both early and late stages of malignant progression. Importantly, as shown in FIG. 9, the C24:1(OH) and C24:0(OH) sulfatide were not detected in normal ducts, islet cells, and in particular, ducts in chronic pancreatitis, a frequent mimic of neoplastic ducts observed in PDAC. (FIG. 9). Although mass spectrometry-based approaches have been previously proposed for surgical margin evaluation in pancreatic cancer resections, the use of sulfatide profiling in this context has, to-date, not been explored.

### Example 5—Rigor of Prior Published Research

High sulfatide levels are a frequently observed phenomena in cancer cell lines and tumor tissues, including lung adenocarcinoma, gastric, kidney, ovarian, breast, and colorectal cancer and elevated sulfatide levels in ovarian and colorectal tumors is associated with poor survival. Similarly, increased tumor expression of UGT8 was shown to be associated with aggressive disease and prognostic for lung metastasis in patients with breast cancer. Yet, mechanistic understanding of deregulated sulfatide in the context of early neoplasia has not been explored. Prior studies suggest that sulfatide is a native ligand for L- and P-selectin, which plays a role in facilitating disease progression, metastasis, and immune modulation. For example, binding of sulfatide to L-selectin was shown to up-regulate expression of the chemokine co-receptor CXCR4 surface expression CD4+ T cells. Other chemokines including CCL2, CCL3, CCL4, CXCL8, and CXCL12 have also been reported to selectively bind sulfatide, collectively suggesting that sulfatide may promote migration of lymphocytes to cancer cells abundance in sulfatide. Moreover, sulfatide has been shown to directly bind receptors on macrophages, resulting in enhanced TGF-β1 and IL-6 secretion, and P-selectin expression. Treatment of antigen-presenting cells with C24:1 sulfatide increased expression of indoleamine 2,3-dioxygenase 1 (IDO1), a rate-limiting enzyme in the kynurenine pathway that is involved in tumor immune suppression. Collectively, these findings hint at a role of sulfatide in modulating the immune response, an aspect that has not been unexplored in the context of pancreatic precursor lesions and in disease progression.

### Example 6—Cross Species Functional and Perturbation Studies on Role of Sulfatides in IPMN Pathogenesis

Rationale: Preliminary studies demonstrated that hydroxylated sulfatide are highly specific to human IPMN epithelium (FIG. 2). MALDI-based imaging of tissues from Kras:Gnas mice also showed hydroxylated sulfatides to be specifically enriched in areas with high-grade dysplasia (FIG. 3), indicating that elevated sulfatide metabolism as a prominent metabolic feature associated with IPMN development and potentially malignant progression. To-date, the cell autonomous role(s) of sulfatide in IPMN remain unknown. Here, interrogation and elucidation of cancer cell dependence and functional relevance of sulfatide in the context of IPMN will be performed using advanced in vitro model systems including patient-derived organoids (PDOs) and genetically engineered murine organoid and cell culture systems.

### Approach:

Patient-derived organoids (PDO): Independent organoids have been generated from fresh biospecimens collected from surgically resected IPMNs.

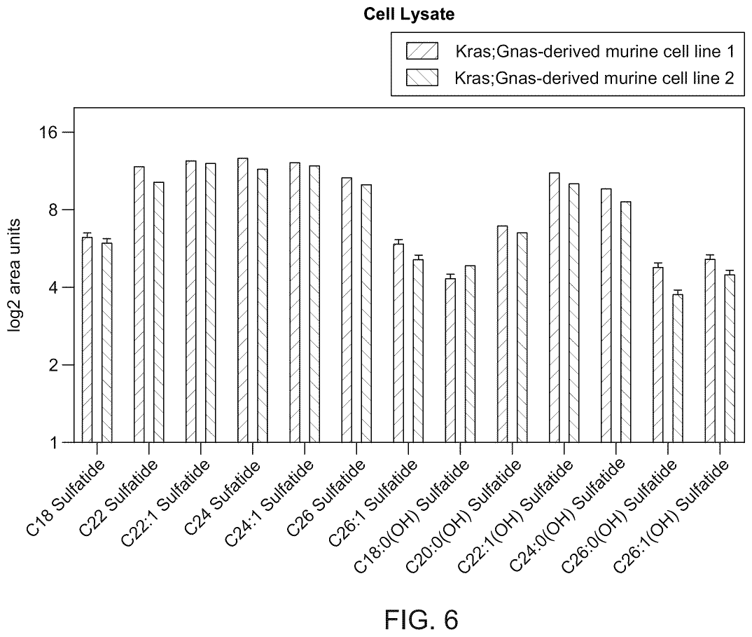

Kras;Gnas-derived murine organoids (MDO) and cell lines: Several organoids and cell lines with Dox-inducible mutant GNASR201C that were derived from Kras;Gnas mice at early stages of lesion development have also been generated. Lipidomic profiling of two of the Kras;Gnas cell lines confirmed sulfatides, including the 24:0(OH) sulfatide, to be readily detectable (FIG. 6), thus making these model systems ideal for studying the biological consequence of increased sulfatide biosynthesis in the context of IPMN.

Experimental Design: Initially, the already established PDOs (n=4) will be used to generate UGT8−/−, Gal3St1−/−, and double UGT8−/−/Gal3St1−/− knockouts using CRISPR/Cas9 technology (Santa Cruz Biotechnology; sc-405110, and sc-407936) to attenuate sulfatide biosynthesis. Independently, knockout of ARSA (Santa Cruz Biotechnology; sc-404921), the primary arylsulfatase that catabolizes sulfatide, to promote accumulation of sulfatide will be done. Knockout efficiency will be confirmed via qPCR and immunoblots. In parallel, UGT8−/−, Gal3st1−/−, UGT8−/−/Gal3st1−/−, and Arsa−/− knockouts (Santa Cruz Biotechnology; sc-423604, sc-424771, and sc-425201) will also be generated in n=4 independent MDOs and n=4 independent Kras;Gnas cell lines. Phenotypic evaluation: Phenotypic effects of single and double UGT8 and Gal3st1 KO will be assessed, as well as ARSA KO on cell proliferation, cell migration and invasion, clonogenic capacity and anchorage-independent growth. Using organoids, growth, viability, and credentialed markers of differentiation (expression of Aquaporins, CFTR, and apomucins) will be evaluated.

To evaluate the acute phenotypic effects of perturbing sulfatide metabolism, silencing RNAs (siRNAs) against UGT8, Gal3st1, and ARSA (ThermoFisher) will be used, as well as small molecule inhibitors of UGT8 (UGT8-IN-1 (MedChemExpress; Cat. HY-131703) and zoledronic acid (ZA) (Sigma Aldrich; Cat. SML0223)). The structures of these inhibitors are as follows:

UGT8-IN-1—[1-[3-(methylcarbamoyl)-7-(trifluoromethyl)thieno[3,2-b]pyridin-5-yl]piperidin-4-yl]N-[(2S)-1-(trifluoromethoxy)propan-2-yl]carbamate

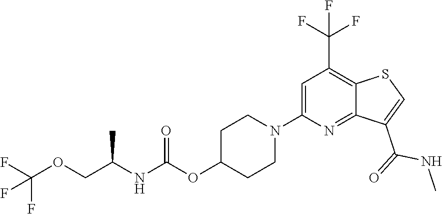 [SMILES: CNC(=O)c1csc2c(C(F)(F)F)cc(N3CCC(OC(=O)N[C@H](C)COC(F)(F)F)CC3)nc12]

Zoledronic acid—[1-[3-(methylcarbamoyl)-7-(trifluoromethyl)thieno[3,2-b]pyridin-5-yl]piperidin-4-yl]N-[(2S)-1-(trifluoromethoxy)propan-2-yl]carbamate

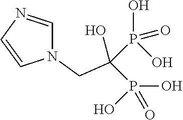 [SMILES: O=P(O)(O)C(O)(Cn1ccnc1)P(=O)(O)O]

For these experiments, additional mechanism(s) of apoptosis using flow cytometry (e.g., Annexin V staining) will be evaluated, as well as immunoblots for apoptotic markers (e.g., caspase-3 and gamma-H2AX). Alternative mechanisms of cell death (i.e., necrosis and ferroptosis) will also be considered. Rescue experiments will be performed through reintroduction of synthetic self-assembled lipid particles (SSALPs) enriched in deuterium-labeled C24:1 galactosylceramide-d7 (Avanti Polar Lipids, #860737) to UGT8 knockdown (KD) cells; stable-isotope resolved lipidomic analyses will be performed to ensure cell uptake and conversion to sulfatide. For Gal3st1 KD cells, SSALPs enriched in C24:1 sulfatide-d7 (Avanti Polar Lipids, #860736) will be reintroduced. The above experiments will be performed in low fetal bovine serum (FBS) or delipidated-FBS containing to mitigate potential cell scavenging of extracellular sphingolipids present in FBS.

Mass spectrometry-based molecular profiling including proteomics, lipidomics, and stable-isotope resolved metabolomics: Untargeted lipidomic analyses of sphingolipids, glycosphingolipid (e.g., galactosylceramides), and sulfatide levels in whole cell lysate extracts. Using a novel density-floatation based approach for isolating highly enriched subcellular compartments including the cell membrane, cytosol, mitochondria, endoplasmic reticulum, Golgi apparatus, and nucleus, changes in sphingolipid and sulfatide metabolism as the subcellular level will additionally be evaluated, in order to gain insights into trafficking and localization. In parallel, in-depth proteomic and phospho-proteomic analyses on whole cell lysate and subcellular compartments will be performed to identify candidate molecular signaling pathways regulated by sulfatide. The resulting molecular profiles will be explored for differential signatures; integrated enrichment analyses (e.g., Ingenuity Pathway Analyses, ActivePathways, Metaboanalyst) will be used to identify key targetable molecular machinery and underlying signaling pathways linked with dysregulated sulfatide metabolism. High priority pathways will be validated using immunoblots, and further explored through genetic manipulation experiments (e.g., SiRNAs, small molecule inhibitors).

Lipidomic analyses: Lipidomic analyses and stable-isotope resolved lipidomic analyses will be conducted on Xevo GS-X2 quadrupole time-of-flight (TOF) mass spectrometers (MS) using a 2D column configuration as described in the art. Peak picking and retention time alignment of LC-MS and MSe data will be performed using Progenesis QI (Nonlinear, Waters). Data processing and peak annotations will be conducted with an in-house automated pipeline. Annotations are determined by matching accurate mass and retention times using customized libraries created from authentic sphingolipid and sulfatide standards and by matching experimental tandem mass spectrometry data against reference spectra. Proteomics analyses: Total cell extract (TCE) of cell lines and organoids will be lysed with 2% OG buffer (PBS, HEPES, Tri-HCl), reduced (TCEP-HCl) and alkylated (2-Chloro-N,N-diethylacetamide) followed by trypsin digestion. Phosphopeptides are further enriched by affinity methods using an anti-phospho-tyrosine (pY) antibody, e.g., pY100 (Cell Signaling Technology), 4G10 (Millipore) and/or TiO2 column (GL Sciences). Recovered phosphopeptides are desalted by reversed phase desalting column (GL-Tip SDB, GL Sciences) and peptides are subsequently analyzed by data-dependent or data-independent scan using a WATERS Synapt G2-Si ion-mobility quadrupole time-of-flight (TOF) mass spectrometer (MS). The acquired mass spectra RAW data is processed and searched against Uniprot Human database through ProteinLynx Global Server (PLGS, Waters) considering Met:Oxidation (+15.994 Da), Ser, Thr, and Tyr: phosphorylation (+79.966 Da) as variable modifications and Cys: diethylacetamide (+113.084 Da) as fixed modification with False Discovery rate 4%.

Statistical considerations and scientific rigor: All in vitro experiments described above will be conducted in triplicates of three independent experiments. Statistical considerations will be made using Tukey's or Dunn's multiple comparison tests depending on data distribution; Student T-tests will be used for in vitro small molecule inhibition experiments. Statistical significance will be determined at a 2-sided p<0.05. Sex as a biological variable will be addressed by using organoids that are evenly balanced from male and female IPMN patients (as well as mice for cross species studies).

### Evaluation of the Tumor-Promoting Functions of Sulfatide Using In Vivo CRISPR Technologies.

Rationale: Preliminary investigations demonstrate that the selective enrichment of sulfatide can be observed at the earliest presentation of pre-neoplastic lesions and persists through progression to carcinoma, suggesting that sulfatides are metabolic determinants associated with tumorigenesis. These effects likely extend into the tumor milieu and host immune interface. For instance, prior studies have shown that immunosuppression by sulfatide reactivated type II natural killer T-cells (NKT) is mediated by IL-13 that results in the generation of tolerogenic tolerogenic dendritic cells (DC) and activation of myeloid-derived suppressor cells (MDSC). Additionally, type II NKT cells limit the anti-tumor function of type I NKT cells. This cross-regulation further leads to suppression of anti-tumor activity of T cells and induces an increase in the function of Treg. Thus, it was hypothesized that sulfatide plays a tumor-promoting role in the progression of pancreatic cystic lesions in part by affecting the tumor microenvironment architecture and modulating the tumor immune repose.

To investigate the role(s) of sulfatide in promoting pancreas tumor formation and progression and evaluates its impact on the nascent tumor microenvironment in vivo, an autochthonous mouse model system and novel CRISPR-based techniques will be used to either knockout or activate sulfatide metabolizing enzymes (Ugt8 and Gal3st1) in the pancreas.

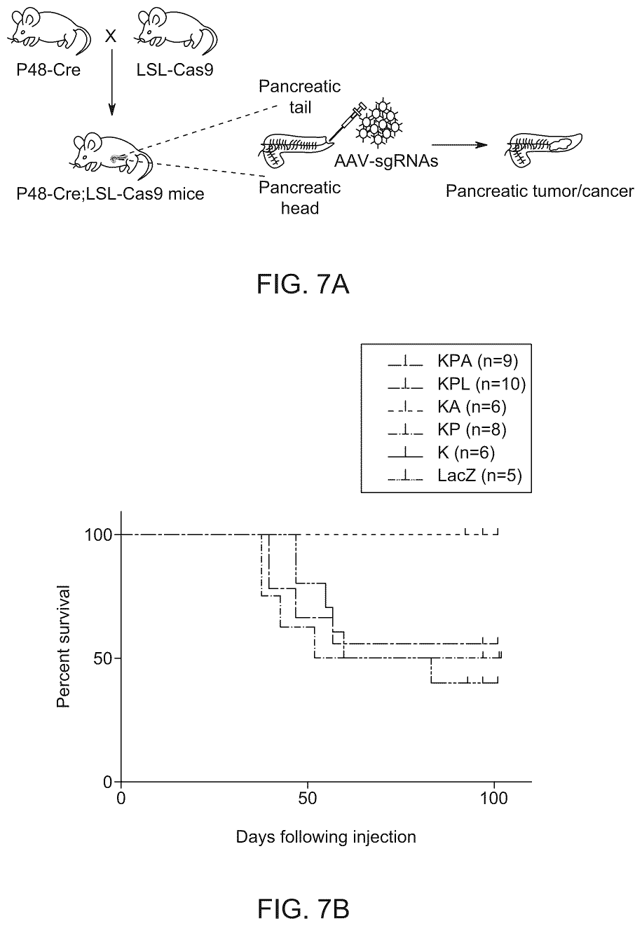

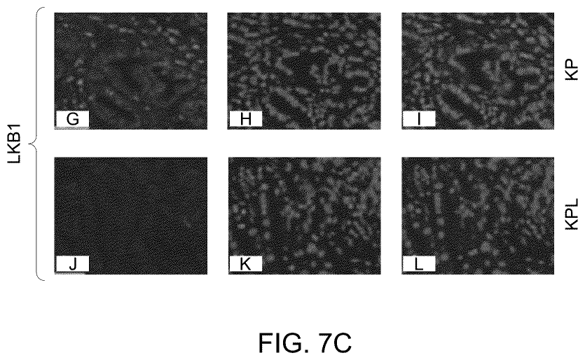

Approach for in vivo CRISPR-KO: A pipeline was established for generating pancreas tumors with programmable genetic knock-out using CRISPR/Cas9 technology. By treating Ptf1a^{Cre}; R26^{LSL-Cas9} mice with adeno-associated virus (AAV) encoding for Kras sgRNA and Kras^{G12D} HDR-donor sequence, in addition to sgRNA targeting one or more genes of interest (GOIs), pancreatic tumors with customized gene deletions can be rapidly generated. Using this approach, pancreas tumor harboring genetic deletion of Trp53, Arid1a and/or Lkb1 were generated, in addition to expressing oncogenic Kras^{G12D} (FIG. 7A). Genetic deletion of these tumor suppressors was found to accelerate tumor formation (FIG. 7B) and result in loss of protein-level tumor suppressor expression (FIG. 7C). Here, this pipeline will be used in combination with the IPMN model to generate cystic lesions with genetic knock-out of enzymes (UGT8 and Gal3St1) required for sulfatide production.

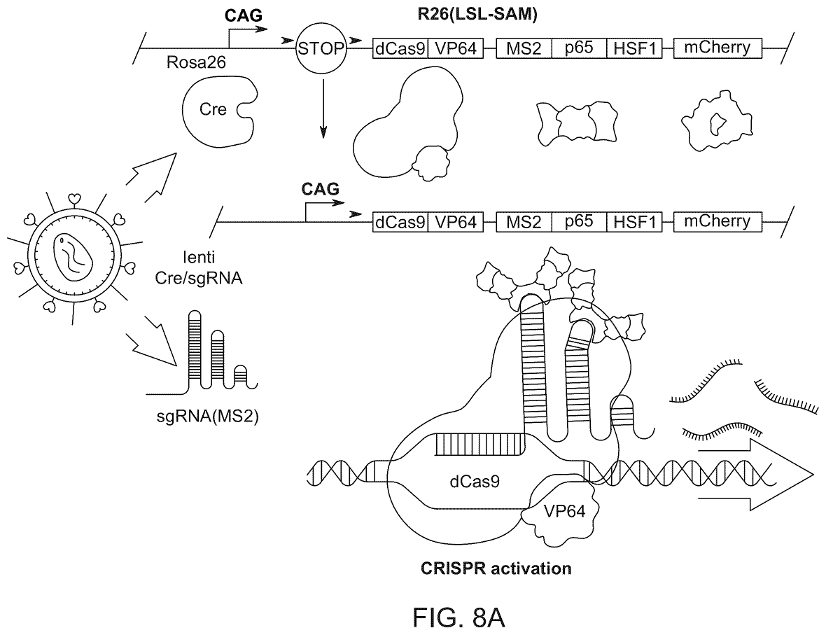

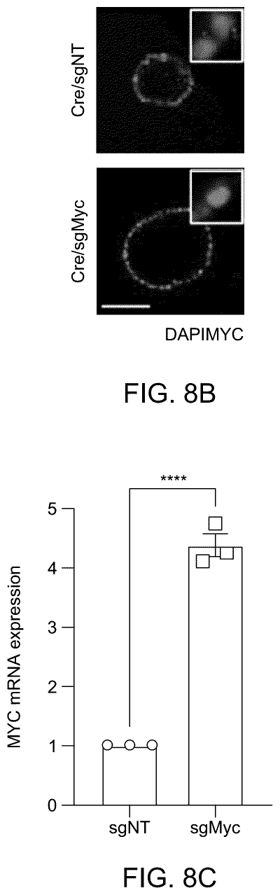

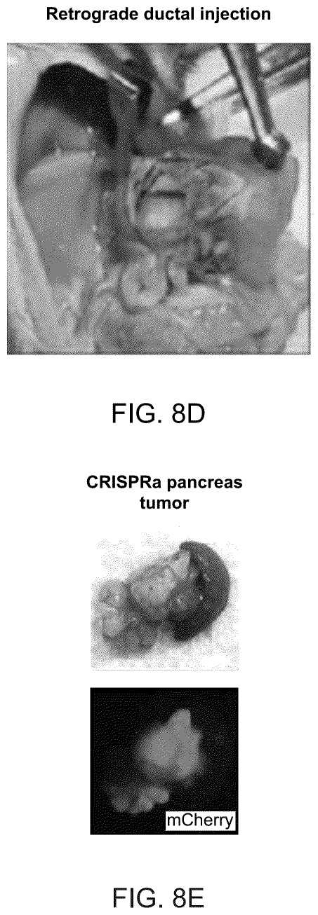

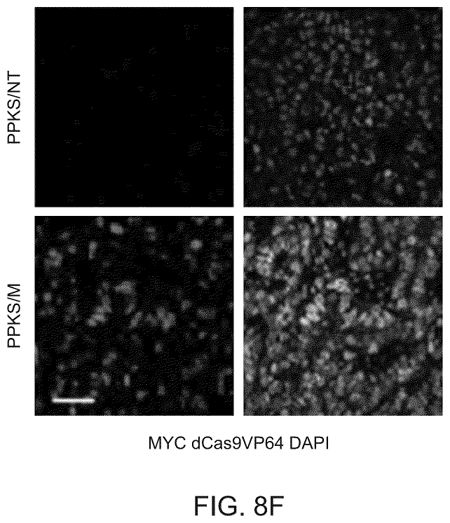

Approach for in vivo CRISPR activation: To allow for programmable gene activation in vivo, a CRISPRa-competent mouse model (the R26LSL-SAM mouse) was established that harbors conditional expression of the factors necessary for in vivo CRISPR activation (dCas9-VP64 and MS2-p65-HSF1), and a lineage label (mCherry). When used in conjunction with MS2-modified single guide RNAs (sgRNA(MS2)) and Cre-recombinase encoding lentivirus, programmed gene activation can be triggered in virtually any tissue or cell type (FIG. 8A). As a proof-of-principle, it has been shown that transduction with Myc-targeting sgRNA lentivirus drives transcriptional and protein-level activation of MYC in pancreatic organoids derived from R26^{LSL-SAM} mice (FIG. 8B). This CRISPRa-competent mouse model was combined with conditional Trp53 loss (Trp53^{fl/fl}) and oncogenic Kras (Kras^{LSL-G12D}) expression to create autochthonous models for lung and pancreas cancer with programmable gene activation using nasal instillation or retrograde ductal injection (FIG. 8D) of Cre/sgRNA(MS2) lentivirus, respectively. Using this model, the oncogenic and immunosuppressive functions of Myc and Yap1 in lung and pancreas adenocarcinoma was investigated (FIG. 8E-F).

The present study proposed to harness the power of the in vivo CRISPR KO and CRISPRa models to comprehensively investigate the tumor promoting functions of sulfatide in IPMN progression.

Experimental design: Established programmable in vivo CRISPR knock-out and activation platforms will be utilized to evaluate the tumor promoting function of sulfatide in pancreatic cancer precursor lesions. The sulfatide production in the pancreas will be modulated during oncogenesis by knocking out (KO) two key enzymes required for sulfatide production (Ugt8 and Gal3st1) in conjunction with expressing oncogenic Kras^{G12D} and Dox-inducible Gnas^{R201C} in vivo. The tumor-promoting function of sulfatide will also be investigated by over-expressing Ugt8 and Gal3st1 in addition to oncogenic Kras^{G12D} in the pancreas.

In vivo CRISPR KO: To decrease the sulfatide production during IPMN formation, R26^{LSL-rtTA/LSL-Cas9}; Tg(Gnas-R201C); Ptf1a^{Cre}; (LGCC) mice will be treated with AAV encoding for Kras sgRNA/G12D HDR, and Ugt8a, Gal3St1 or non-targeting sgRNA though retrograde ductal injections. In these mice, AAV-driven expression of Kras-targeting sgRNA in combination with Kras^{G12D} HDR donor sequence will result in oncogenic Kras^{G12D} being expressed sporadically throughout the Cas9-expressing exocrine (Ptf1a+) pancreas lineage. In the same cells, UGT8 and/or Gal3St1 will be knocked out through the action of the AAV-encoded gene-targeting sgRNA, whereas non-targeting sgRNA will be used to generate control tumors. With doxycycline treatment oncogenic Gnas^{R201C} will be expressed in addition to oncogenic KrasG12D leading to progression through a cystic/IPMN-like pathway. Table 1 shows the 4 mouse groups used in these experiments.

TABLE 1
Mouse Groups Used in Study

| Mouse | | | |
|---|---|---|---|
| Genotype | Virus | Treatment | Comment |
| LGCC | AAV: sgKras/G12D | Dox+ | Control, IPMN |
| | HDR, sgNT | | |
| LGCC | AAV: sgKras/G12D | Dox+ | UGT8 KO, IPMN |
| | HDR, sgUGT8 | | |
| LGCC | AAV: sgKras/G12D | Dox+ | Gal3St1 KO, IPMN |
| | HDR, sgGal3St1 | | |
| LGCC | AAV: sgKras/G12D | Dox+ | UGT8/Gal3St1 |
| | HDR, sgUGT8, sgGal3St1 | | KO, IPMN |

Endpoint analyses: The primary outcome of interest will be overall survival (long-term) and tumor development and growth (short-term). For long-term survival experiments (n=10 per cohort; n=40 mice in total), animals will be monitored daily and euthanized if any mice become moribund. For short-term experiments, pancreas will be collected from an independent set of mice at 6, 12, and 18 weeks of CRISPR KO (n=4 mice per group per time point). Correlatives: Pancreas tissues will be used for detailed histopathology and quantitative assessment of precursor lesions and cancer, spatial distribution and levels of sulfatide via MALDI-MS imaging, and assessment of the tumor microenvironment (TME). EDTA plasma will be collected for multi-plexed analysis of cytokines and chemokines. Spatial Transcriptomics (ST) using the Visium platform (10× Genomics, CA) will be performed to conduct a characterization of the spatio-molecular components of the tumor, including assessment of UGT8, GAL3ST1, and other transcripts of interest, and evaluation of the tumor microenvironment composition and organization. Spatial transcriptomic data will be deconvolved using the Robust Cell Type Deconvolution (RCTD) method to infer cell type proportions for each of the spots using a single cell RNA sequencing reference dataset to evaluate changes in cell composition associated with KO of Ugt8 and Gal3st, such as cancer-associated fibroblast (CAF) or NK cell levels. Multiplex immunofluorescence panels will be used to validate findings of interest based on the ST data, particularly phenotyping of the immune microenvironment using comprehensive antibody panels. Markers to be included for multiplex immunofluorescence or flow cytometry include:

(1) Natural killer T-cells (NKT) cells: Type I NKT cells (CD1d-αGalCer tetramer⁺TCRβ⁺/CD3⁺) Type II NKT cells (CD1d-αGalADAG/CD1d-Sulfatide tetramer+TCRβ+/CD3+); cytokines: IFN-γ, IL-4, IL-2, IL-13.

(2) Dendritic cells (DC): CD11c, CD11b, B220, PDCA-1, MHC-II, CD80, CD86, IL-12, IL-10; mDC (CD11^{hi}CD11b⁺), pDC (CD11c^{int}B220⁺/PDCA⁺)

(3) Macrophages: CD11b, F4/80, MerTK, CD68, CD369, MHC-II, CD80, CD86, CD163, CD206, IL-10, IDO distinguish M1, M2 and TAM.

(4) T Cells: CD3, CD4, CD8, CD25, T-bet, Foxp3, IFNγ, and IL-2 to distinguish CD8, CD4 and Treg cells.

Statistical considerations and scientific rigor: As sex is a biological variable, mice will be randomized as close as possible to a 1:1 ratio for long- and short-term experiments. Considering a 100% probability of mice developing pancreatic cancer, with a sample size of 10 mice per group, assuming a Hazard Ratio of 0.1 for the experimental group (UGT8 KO; Gal3St1 KO; UGT8/Gal3St1 KO) and a probability of death of 80% in the control group and 20% in the experimental group over the period of follow-up, there will be 83% power to obtain a statistically significance difference in survival outcomes using log-rank test controlling for significance at a level of 0.05 and assuming the test is one-sided. This power calculation was derived using the Rosner approach. A sample size of 4 mice per experimental group (UGT8 KO; Gal3St1 KO; UGT8/Gal3St1 KO) versus control group yields power≥82% at a significance level (α) of 0.05 for the primary outcome of interest (tumor volume) assuming that the effect size is ≥2.1 using Wilcoxon-Mann Whitney test. For survival analyses, Log-rank (Mantel-Cox) tests or Gehan-Breslow-Wilcoxon tests will be used, depending on proportionality of the Cox proportional hazard model assumptions. Continuous variables will be compared with the Student's t-test and categorical variables with the χ² test. Nonparametric tests will be used to compare differences if appropriate. The Benjamini-Hochberg (BH) method will be used to adjust p-values for multiple-comparison testing.

Anticipated results and alternative approaches: It is anticipated that in vivo KO of Ugt8 and/or Gal3st1 will lead to decreased sulfatide production, remodel the IPMN/tumor and immune microenvironment, and retard IPMN progression. While sporadic Ugt8 and Gal3st1 KO in the pancreas is expected to be well tolerated, it is possible that knock-out cells may apoptose or be cleared by the immune system prior to IPMN formation. If such difficulties are encountered, or other issues generating IPMNs or tumors using this approach, the Ugt8 and/or Gal3st1 KO LGKC (IPMN) organoids described herein will be used, followed by orthotopic transplantation into syngeneic mice. This would allow addressing the same questions outlined in the aim above in a more tractable, lower-risk experimental framework.

In vivo CRISPR activation: It will also be investigated if sulfatide production accelerates pancreas tumor formation. To increase the sulfatide production during tumor formation, Kras^{LSL-G12D}; R26^{LSL-SAM} (KS) mice will be treated with Ugt8a, Gal3st1, or non-targeting sgRNA(MS2)/CMV-Cre lentivirus through retrograde ductal injection. Using this approach, oncogenic Kras will be expressed only in transduced and Cre-recombined cells in the pancreas, and over-expression of Ugt8 and Galst1 will be driven by the expression of the SAM construct and gene-targeting sgRNA(MS2). Table 2 shows the 4 mouse groups used in these experiments.

TABLE 2
Mouse Groups Used in Study

| Mouse | | |
|---|---|---|
| Genotype | Virus | Comment |
| KS | Lenti: CMV-Cre, sgNT(MS2) | Control |
| KS | Lenti: CMV-Cre, sgUGT8(MS2) | UGT8 OE |
| KS | Lenti: CMV-Cre, sgGal3St1(MS2) | Gal3St1 OE |
| KS | Lenti: CMV-Cre, sgUGT8(MS2), | UGT8/Gal3St1 OE |
| | sgGal3St1(MS2) | |

OE, overexpression

Endpoint analyses: The primary endpoints will be as described above. For long-term survival experiments, n=20 mice per group (n=80 mice total) will be used. Animals will be monitored daily and euthanized if any mice become moribund. For short-term experiments, pancreas will be collected from an independent set of mice at 6, 12, and 18 weeks of CRISPR activation (n=8 mice per group per time point). Correlatives: Pancreas tissues will be used for detailed histopathology and quantitative assessment of precursor lesions and cancer, spatial distribution and levels of sulfatide via MALDI-imaging, and assessment of the tumor microenvironment (TME) will be performed as described above. EDTA plasma will be collected for multiplexed analysis of cytokines and chemokines.

Statistical considerations and scientific rigor: As sex is a biological variable, mice will be randomized as close as possible to a 1:1 ratio for long- and short-term experiments. Considering a 50% probability of mice developing pancreatic cancer, a sample size of 20 mice per group, assuming a Hazard Ratio of 0.1 for the experimental group (UGT8 OE; Gal3St1 OE; UGT8/Gal3St1 OE) and a probability of death of 20% in the control group and 80% in the experimental group over given period of follow-up, will yield>83% power to obtain a statistically significance difference in survival outcomes using log-rank test controlling for significance at a level of 0.05 and assuming the test is one-sided. A sample size of 8 mice per experimental group (UGT8 OE; Gal3St1 OE; UGT8/Gal3St1 OE) versus control group has power≥82% at a significance level (α) of 0.05 for the primary outcome of interest (tumor volume) assuming that the effect size is ≥2.1 using Wilcoxon-Mann Whitney test. For survival analyses, Log-rank (Mantel-Cox) tests or Gehan-Breslow-Wilcoxon tests will be used, depending on proportionality of the Cox proportional hazard model assumptions as described above. Continuous variables will be assessed using Student's t-test; categorical variables using the χ² test. Non-parametric tests will be applied if appropriate. P-values will be adjusted for multiple comparison testing using the BH method.

Anticipated results and alternative approaches: It is anticipated that in vivo activation of UGT8 and/or Gal3St1 will increase sulfatide production, remodel the tumor and immune microenvironment, and accelerate tumor progression. Since lipid modifications, such as glycosylation and sulfonation, result from complex multistep enzymatic processes with branching points (i.e., shunting into alternative metabolic pathways), it is possible that activation of UGT8 and/or Gal3St1 expression does not increase sulfatide production in vivo. If increased sulfatide deposition is not detected in UGT8/Gal3St1 over-expressing pancreata, UGT8 and/or Gal3St1 will be overexpressed in Kras^{G12D};Gnas^{R201C} organoids, followed by orthotopic transplantation into syngeneic mice in the absence of Dox supplementation (thus GNAS^{R201C} is not activated).

Evaluation of whether small molecule inhibition of UGT8 is a viable strategy for cancer interception in IPMN mice.
Rationale: Preliminary studies provide substantive evidence that sulfatide, as well as corresponding gene transcripts of enzymes involved in sulfatide metabolism, are specifically enriched in pancreatic precursor lesions and carcinomas (FIGS. 2-3, 5). Lipidomic analyses of Kras;Gnas cells similarly demonstrate sulfatide to be readily detectable (FIG. 6). It was hypothesized that enhanced sulfatide production in pancreatic precursor lesions and carcinomas presents a targetable metabolic vulnerability that can be exploited for anti-cancer treatment.

Approach: The Ptf1a^{Cre}; Kras^{LSL-G12D}; R26^{LSL-rTA}, Tg(Gnas-R201C) (Kras;Gnas) mouse model will be used to test the efficacy of targeting UGT8 for attenuating malignant progression of cystic lesions. Nearly all Kras;Gnas mice develop cystic lesions as early as 6 weeks post Dox induction and approximately 25% of them will progress to invasive PDAC at a median of 21 weeks post Dox induction. Studies will be initiated using 3 mg/kg of UGT8-IN-1 (MedChemExpress; Cat. No. HY-131703), an orally available inhibitor of UGT8, via daily oral gavage. Prior mouse studies have shown that 3 mg/kg results in >90% inhibition of incorporation of ¹³C-Gal into GalCer and sulfatide; with an effective dose (ED)₅₀ of 1 mg/kg. Treatment (n=16 mice per group) will be initiated starting at 6 weeks post Dox induction when mice develop cystic lesions and treatment will be continued for 15 weeks to reach the 21-week cutoff. Endpoint analyses: The primary outcome of interest will be development of precursor lesions and invasive carcinoma between treated vs. untreated (saline control) mice. The extent of precursor lesions and tumors and respective size will be measured by ultrasound at 5, 10, and 15 weeks post start of intervention. Animals will be monitored for signs of toxicity, including body weight, altered behavior, distress and morbidity. The dosage of UGT8-IN-1 (e.g., 1 mg/kg) will be reduced if toxicity is observed. At necropsy, the body weight will be recorded for each mouse. Correlatives: Pancreas tissues will be collected from mice following intervention and used for detailed histopathology and quantitative assessment of precursor lesions and cancer, as well as assessment of the TME as described herein. Histopathological examination of major organs (e.g., brain, liver, and kidney) and blood chemistry tests will be performed to assess for signs of treatment-associated toxicity.

Statistical Considerations and scientific rigor: As sex is a biological variable, mice will be randomized as close as possible to a 1:1 ratio for long- and short-term experiments. A sample size of 16 mice per group is required for the detection of a 20% difference (i.e., p1−p0=0.20) in incidence of cancer with 80% power and assuming that p0=0.25. Note that with the selected sample size, the null hypothesis that p0=0.25 at the α level of significance of 0.1 would be rejected if 2 (out of 16) or less mice progress. Number of lesions developed after treatment will be modeled by Poisson distribution, or negative binomial distribution if appropriate, and compared between treatment groups using generalized linear models. Zero-inflated models will be considered if the drug treatment inhibits tumor formation and results in zero tumor in large proportion of mice. The extent of tumors as measured by ultrasound will be compared using 2-way ANOVA. Data transformation will be considered if the distribution is not normal. Tumor size change will be compared among treatment groups. Descriptive statistics, box plot and plots of the change over time will be used to summarize the tumor size measure. At each time point, two-sample t-test will be used to compare two groups. Data transformations will be considered when the tumor size measure is not normally distributed. Furthermore, mixed effect models will be used to model the changes of tumor size over time and to compare treatment groups in terms of tumor growth rate, where the time-by-treatment interaction will be tested to assess if treatment slows tumor growth. Data transformation will be considered if the distribution is not normal. A non-linear time curve will be considered if appropriate.

Anticipated results and alternative approaches: It is anticipated that UGT8-IN-1 will elicit a potent anti-cancer effect and attenuate the progression of precursor lesions in Kras;Gnas mice. As UGT8-IN-1 can penetrate the blood-brain barrier, animals will be monitored closely for signs of treatment-associated toxicity, including neurotoxicity, and a lower dose of UGT8-IN-1 will be administered if toxicity is observed. Alternative inhibitors of UGT8, such as zoledronic acid, will be also considered. Treatment of Kras^{LSL-G12D}; R26^{LSL-SAM} mice following CRISPR-based activation of UGT8 in pancreas as an alternative model system to demonstrate feasibility of targeting sulfatide metabolism to prevent progression of precursor lesions.

### Example 7—In Vitro Toxicity Assay for Mouse IPMN Cell Lines

In vitro toxicity assays (MTS assays) were performed using three independent mouse IPMN cell lines (LGKC 4301, LGKC 4838, and LGKC 4861). Briefly, each cell line was treated with either vehicle control or the UGT8 inhibitors UGT8-IN-1 or zoledronic acid. Experiments were performed in technical triplicates. Statistical significance was determined by 2-sided Student T-test.

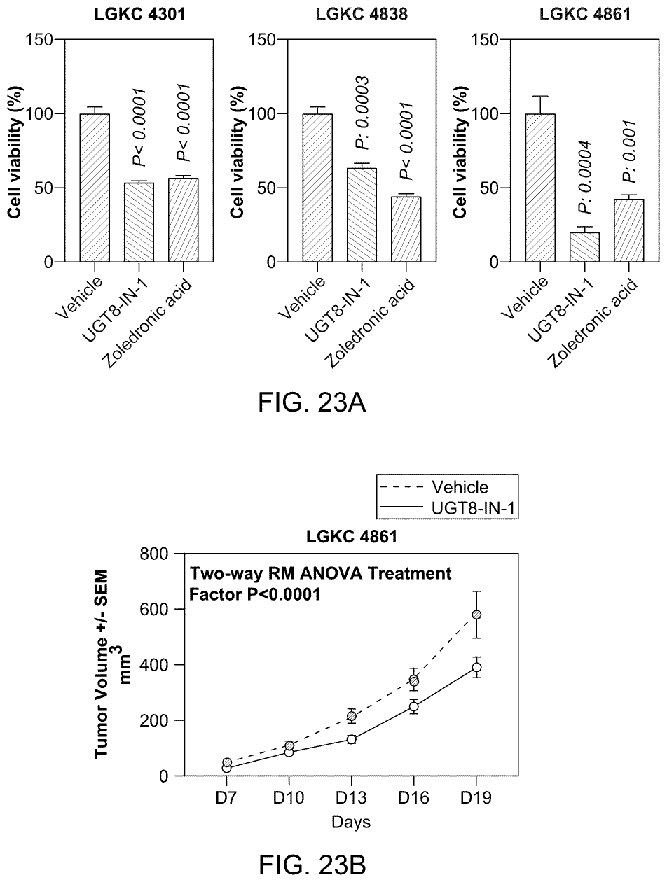

The results demonstrated that UGT8-IN-1 and zoledronic acid performed similarly in LGKC 4301 cells (UGT8-IN-1 was slightly more effective). On the contrary, zoledronic acid resulted in a greater reduction in cell viability than UGT8-IN-1 in LGKC 4838 cells, while UGT8-IN-1 greatly reduced viability of cell line LGKC 4861 compared to zoledronic acid (˜80% and ˜55% reduction in cell viability, respectively) (FIG. 23A).

In addition to in vitro toxicity assays, mouse IPMN cell line LGKC 4861 (p48-Cre; LSL-KrasG12D; Rosa26R-LSL-rtTA-TetO-GnasR201C), for which UGT8-IN-1 was most effective, were implanted subcutaneously in 8-week-old male and female athymic nude mice (1×10⁵ cells per mouse) with doxycycline (200 μg/ml) supplemented in drinking water. UGT8 inhibitor UGT8-IN-1 or vehicle control (saline) was orally administrated every other day (3 mg/kg) from day-8 post cancer cell implantation for a period of 2 weeks. Tumor volume was measured every 3 days. Statistical significance was determined by 2-way repeated measures ANOVA, and treatment factor 2-sided p-value is reported. As shown in FIG. 23B, treatment of mice with the UGT8 inhibitor UGT8-IN-1 resulted in a lower tumor volume compared to vehicle.

### Example 8—MALDI-MS Imaging of Sulfatide Species to Evaluate Intraoperative Tissues from Pancreatic Cancer Resections

Rationale: Surgery remains the only current curative option for patients with resectable pancreatic cancer. To guide surgical resection and ensure adequate removal of the cancer, tissue margins from the pancreatic neck, uncinate process and common bile duct, among others, are commonly assessed by intraoperative frozen section evaluation. However, detection of pancreatic cancer at these margins for intraoperative assessment may be missed with currently used routine frozen section techniques, leading to misguidance on additional surgical need, which leads to an even poorer prognosis for the patient. Consequently, there is a pressing need to incorporate molecular information to improve accuracy of pancreatic cancer margin evaluation and help guide pancreatic cancer resection.

Preliminary studies showed high relative abundance of sulfatide species, particularly C24:1(OH) and C24:0(OH), in the epithelial linings of human and murine IPMN tissue samples (FIGS. 2-3). Sulfatide lipid species were also detected in PDAC tissue areas, both from cases with and without co-occurring IPMN, suggesting sulfatide lipids are present in neoplastic ducts at both early and late stages of malignant progression. Importantly, as shown in FIG. 9, the C24:1(OH) and C24:0(OH) sulfatide were not detected in normal ducts, islet cells, and in particular, ducts in chronic pancreatitis, a frequent mimic of neoplastic ducts observed in PDAC. Building upon this data and capitalizing on the high specificity of sulfatide levels to neoplastic pancreatic lesions, it was proposed to investigate the potential of utilizing sulfatide imaging for margin assessment of pancreatic tissue specimens from surgical resections. Although mass spectrometry-based approaches have been previously proposed for surgical margin evaluation in pancreatic cancer resections, the use of sulfatide profiling in this context has, to-date, not been explored.

Approach: Sample Accrual: Surgical margins will be collected from pancreatic cancer surgical resections as frozen tissue sections under approved protocols. At least 15 positive margins will be collected, including uncinate, bile duct, and pancreatic neck margins, from frozen tissue sections as well as five times the number of negative margins (n=75; 1:5 positive:negative ratio) per year with a projected total of 30 positive and 150 negative margins collected. Additionally, and when amenable, frozen sections from suspicious metastatic nodules found during surgery, such as liver or peritoneal nodules, will also be collected for this study. Diagnosis of metastatic deposits at these sites on intraoperative assessment leads to termination of surgery, reiterating the importance of accurate diagnosis. Thirty frozen sections from suspicious nodules will be collected per year (n=60 total), with an anticipated 50% positivity rate based on historical data. Margin and tumor status will be initially determined based on frozen section evaluation. Full pathological reports obtained from examination of the fixed specimens post-operatively will also be collected. PDAC tissue specimens obtained from surgical resection will be analyzed and processed in the Ahmed Center for Pancreatic Cancer to determine the extent to which sulfatide levels correlate with margin positivity if present. Samples will be cryo-sectioned at 10 μm and stored at −80° C. until MALDI-MS analysis.

MALDI-MS Imaging and Analysis: MALDI-MS matrix application of 9AA (9-Aminoacridine) will be performed using an HTX M5 sprayer (HTX Technologies, NC). Analyses will be conducted in the negative ion mode scanning from m/z 50-2000, using resolution mode on a MALDI SYNAPT G2-Si (Waters, MA). MALDI-MS imaging will be performed at 60 μm spatial resolution. Hematoxylin and eosin staining will be done on both the same and a serial tissue section. Regions of interest, determined by histology as suspicious for cancer, precursor lesions, or pancreatitis, will be selected and their sulfatide levels determined.

Statistical Considerations and scientific rigor: To determine feasibility of sulfatide for detecting pancreatic cancer, various AI approaches will be used, including feedforward and backpropagation neural network with more than 3 hidden layers (to account for edge consideration in the hidden layers), convolutional neural network, recurrent neural network to correctly classify the surgical margins. To evaluate model stability and tuning hyperparameters, k-fold cross validation will be performed. The model with the best performance will be selected, and the model will be considered clinically meaningful if the overall sensitivity is statistically significantly greater than 80% at 100% specificity. Power Calculation: With 30 positive tissues and 150 negative margin tissues, assuming a sensitivity of >97% at a specificity of 100% and controlling Type 1 error at 0.05, there will be >87% power to reject the null hypothesis. In this power calculation, the null hypothesis is the performance of the intraoperative frozen section66 has sensitivity 80% at 100% specificity. Sample size calculation was based on the method that has been developed by Pepe et al. Table 3 shows power estimates at different sensitivity thresholds.

TABLE 3
Power estimates at varying sensitivity thresholds

| | Sensitivity | | | | |
|---|---|---|---|---|---|
| Specificity | 96% | 96.50% | 97% | 97.5% | 98% |
| 100 | 68.8 | 78.2 | 87 | 95 | 99 |

Anticipated results and alternative approaches: MALDI-based imaging of sulfatide in intraoperative tissues from pancreatic surgeries is expected to yield a ‘sulfatide signature’ that can identify positive margins and malignant nodules. For example, the 2 primary sulfatides that show the highest selectivity toward IPMN include, e.g., C24:0(OH) sulfatide and C24:1(OH) sulfatide. Elevation of these 2 sulfatide species is indicative of IPMN and the progression from IPMN to PDAC. Other sulfatide species, such as C16 sulfatide are also fairly specific to IPMN although perhaps to a lesser extent.

In addition to sulfatide, MALDI-imaging will yield other non-sulfatide lipids, thus allowing for additional discovery efforts to derive alternative lipid signatures that may improve accuracy of pancreatic cancer margin evaluation.

### Example 9—ST Analysis Using Sample Cohort

Fresh-frozen IPMN samples collected from surgical resections were evaluated by a pathologist. A cohort of 7 low-grade (LG) IPMN, 3 high-grade (HG) IPMN, and 3 PDAC samples were selected. Samples were sectioned at 10 μm and serial sections were used for ST and MALDI. ST analyses were conducted utilizing the Visium Spatial Gene Expression technology (10× genomics, CA) and sequenced on a NextSeq 550 (Illumina, CA). MALDI imaging was performed on a MALDI SYNAPT G2-Si (Waters, MA) from m/z 50-2000 at high resolution mode. The HTX M5 sprayer (HTX Technologies, NC) was used for matrix deposition of CHCA (a-cyano-4-hydroxycinnamic acid) and 9AA (9-Aminoacridine) for positive and negative ion mode, respectively.

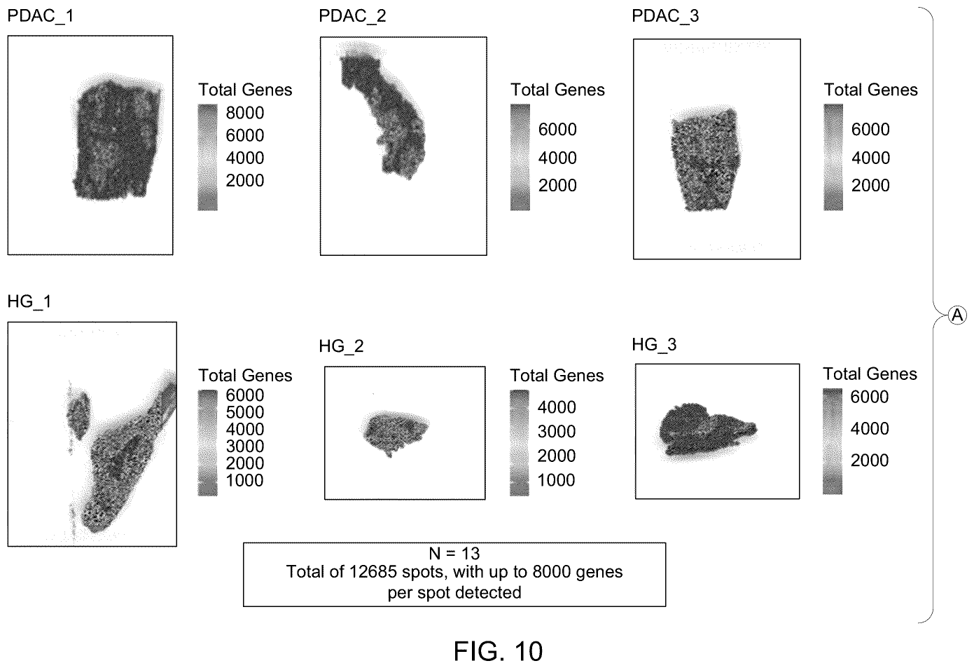

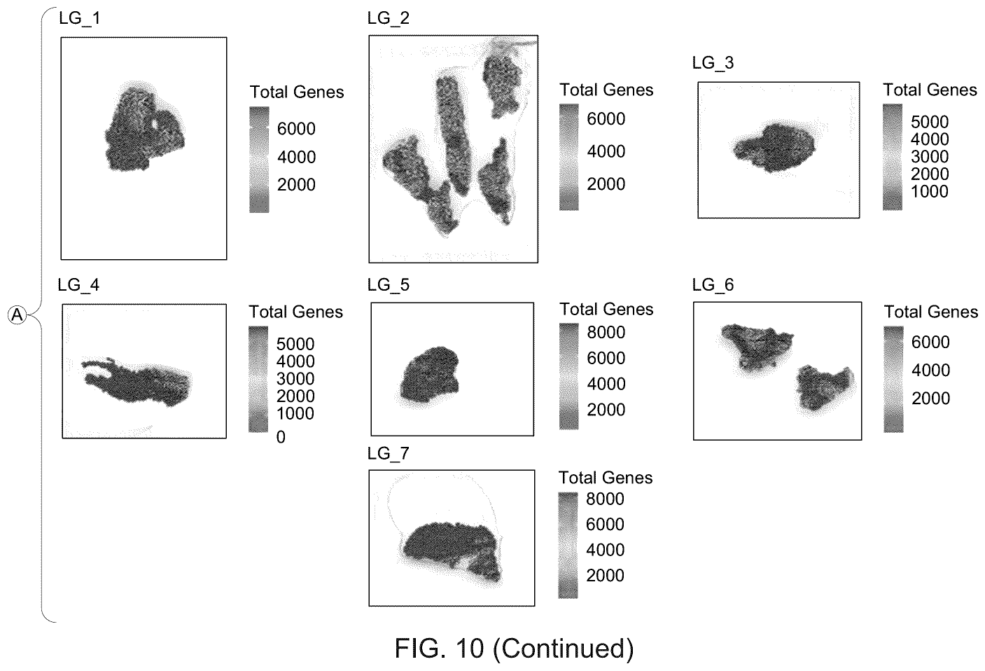

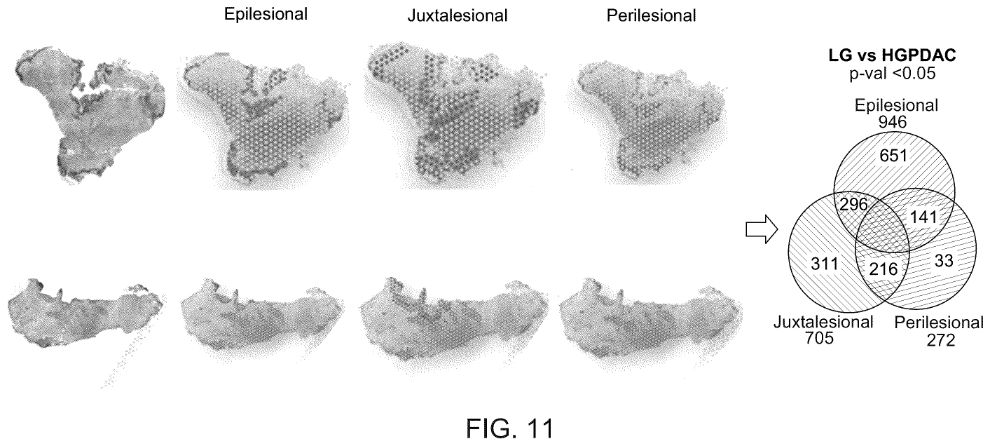

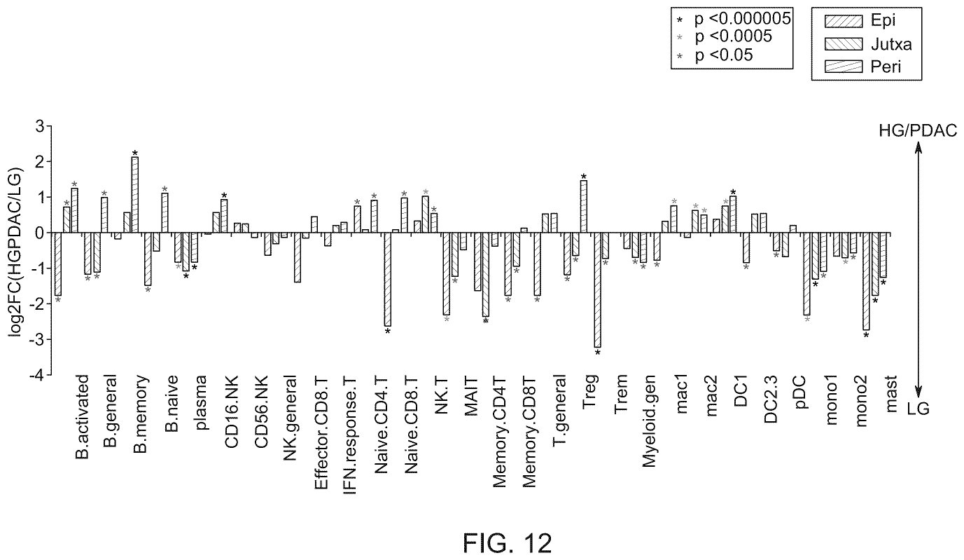

ST analyses yielded spatially resolved expression profiles for up to 8,000 genes per each of the 12,685 spots (50 m) covering the tissue samples. Histologically directed analysis was applied, focusing on areas covering the epithelial lining of the IPMN lesions (“epilesional” areas), as well as the adjacent microenvironment surrounding the lesion (‘perilesional” areas), resulting in the identification of various regional-specific genes associated with grades of dysplasia in IPMN (FIGS. 10-11).

Targeted selection of different areas in the tissue specimens included: (1) Epilesional: outlining of the epithelium of IPMN and PDAC samples; (2) Juxtalesional: adjacent tumor microenvironment (TMM, two spots further); (3) Perilesional: two additional spots out (more like normal stroma); (4) differential expression of the different areas comparing LG vs HG PDAC.

Differences in gene expression, as well as cell composition in the perilesional areas were observed, such as the infiltration of mast cells in LG perilesional areas but not in HG IPMN or PDAC, suggesting a remodeling of the tumor microenvironment associated with IPMN progression. Gene transcripts encoding proteins involved in various metabolic pathways, including fatty acid, glutathione, ether lipid, fructose, glycerophospholipid, and o-glycan metabolism and biosynthesis, as well as glycolysis and gluconeogenesis, were found to be expressed at significantly different levels between LG IPMN and the HG IPMN+PDAC epilesional areas, suggesting a continuous dysregulation of metabolite, lipid, and glycan metabolism driven by neoplastic progression of IPMN. Leveraging the ST data, regions overlapping with histological and transcriptomic profiles of interest were selected from the MALDI-MS images. MALDI-MS imaging detected various lipid species, including small metabolites, fatty acids, lysophospholipids, glycerophospholipids, and sphingolipids. Of note, complex glycosphingolipids, such as sulfatides and cerebrosides were identified at high abundance in the epilesional compartments, particularly in those from HG IPMN and PDAC samples. Interestingly, expression of transcripts for ST3GAL4 (ST3 beta-galactoside alpha-2,3-sialyltransferase 4), responsible for the processing of glycoproteins and glycolipids, was also detected at high levels on ST analysis in those areas. Current efforts are directed towards the evaluation of the spatial colocalization of additional transcripts linked with specific lipid profiles observed in epilesional and perilesional tissue areas both through intra- and inter-patient comparisons.

Single-cell deconvolution was performed with the reference scRNAseq dataset, which allows inference of cell composition. FIG. 12 shows the log 2FC of the spot proportions between LG and HG-PDAC. Cell types showing higher in the lower panel (below zero) indicates higher levels in LG samples, and those showing higher in the upper panel (above zero) indicate higher levels in HG-PDAC samples. There was an overall higher degree of immune infiltration in LG IPMN vs HGPDAC, for example, a higher proportion of mast cells and plasma cells in all 3 compartments.

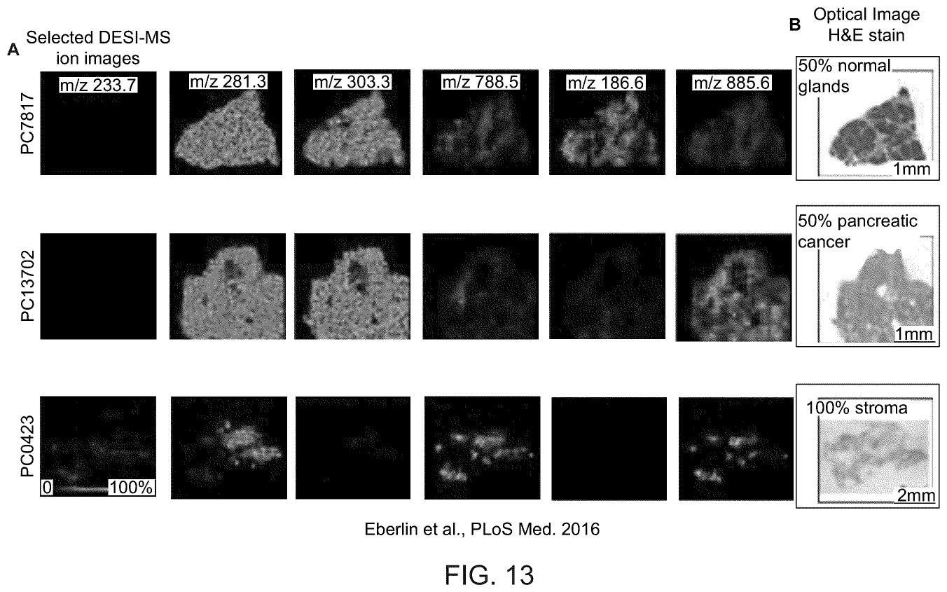

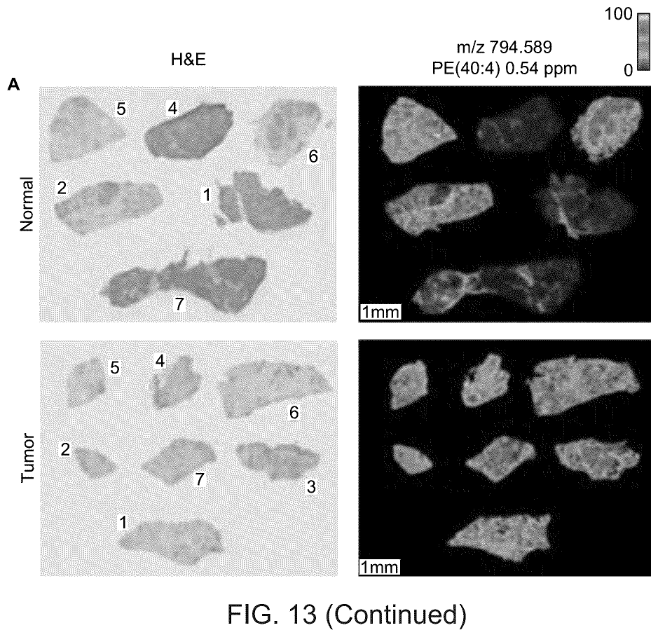

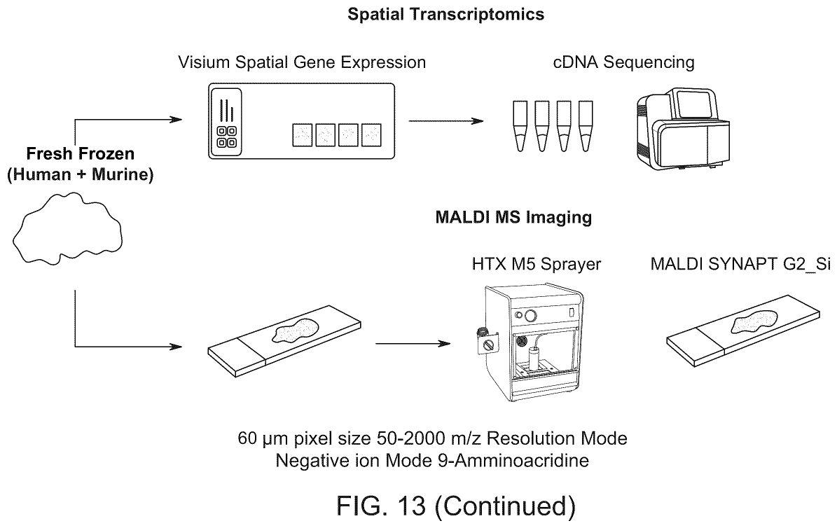

FIG. 13 shows an overview of data from spatially resolved lipid analysis, using serial sections to do MALDI-MS imaging. Previous studies have observed differences in the lipid profiles between tissue types evaluated with mass spectrometry lipid imaging of pancreatic cancer, and the correlation of how those were associated with gene expression profiles was studied. MALDI-MS imaging on a waters SYNAPT G2-Si, at a 60 μm pixel size, and the following parameters: 50-1000 m/z, resolution mode, negative ion mode, 9-amminoacridine.

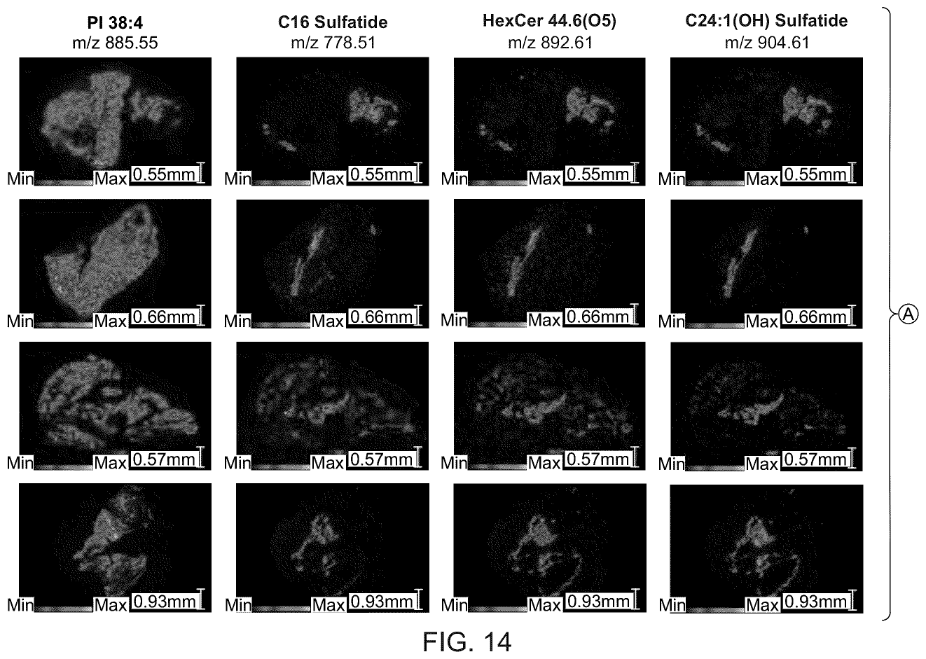

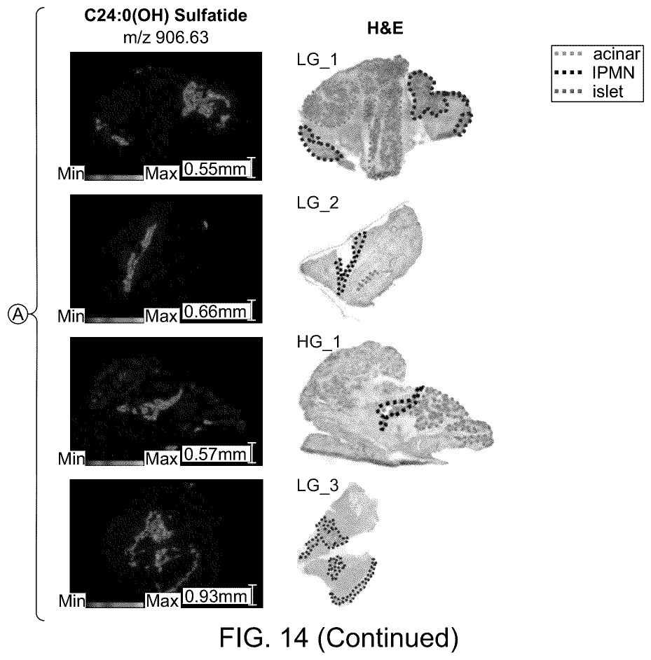

As shown in FIG. 14, MALDI-MS imaging reveals enrichment of cerebroside species in IPMN. Strikingly, high levels of cerebroside and sulfatide species were observed outlining the IPMN and PDAC epithelium in these samples. For example, PI 38:4 showed an unspecific distribution, with detection also in acinar cells, and stroma, etc. However, sulfatide species were exclusively detected in the specific IPMN and PDAC areas.

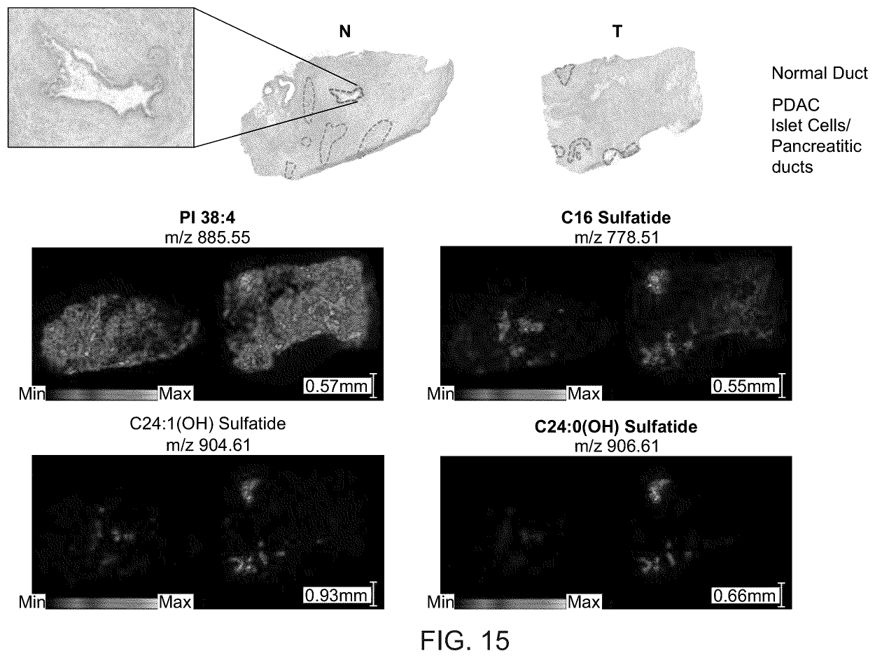

Normal and tumor pairs from the same patient were analyzed. As shown in FIG. 15, there was higher expression of the long-chain hydroxylated sulfatides in the PDAC areas when compared to adjacent stroma and normal ducts. On the other hand, shorter chain sulfatide had similar expression between normal duct and cancer.

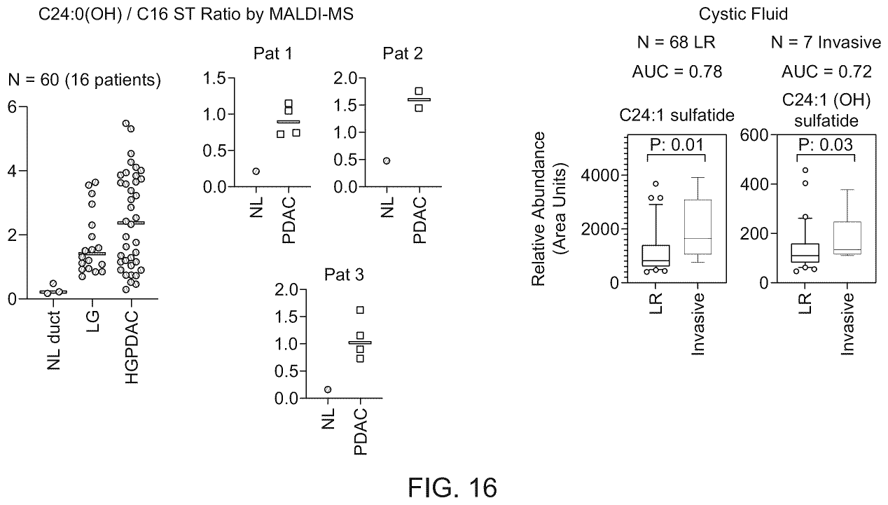

FIG. 16 shows that long-chain hydroxylated sulfatides are elevated in PDAC. To evaluate this trend, the ratio of long to short chain sulfatide was evaluated. From evaluation of 60 ROIs from 16 patients, there is an increase in the ratio of long-chain hydroxylated sulfatides when comparing normal ducts to LG-IPMN and then HG-PDAC. The ratio for 3 patients comparing normal duct and cancer from the same patient had a higher ratio in the cancer samples versus normal ducts. In addition, higher levels of these sulfatides were observed in cystic fluid from patients with higher risk or invasive IPMN vs Low risk (low-grade).

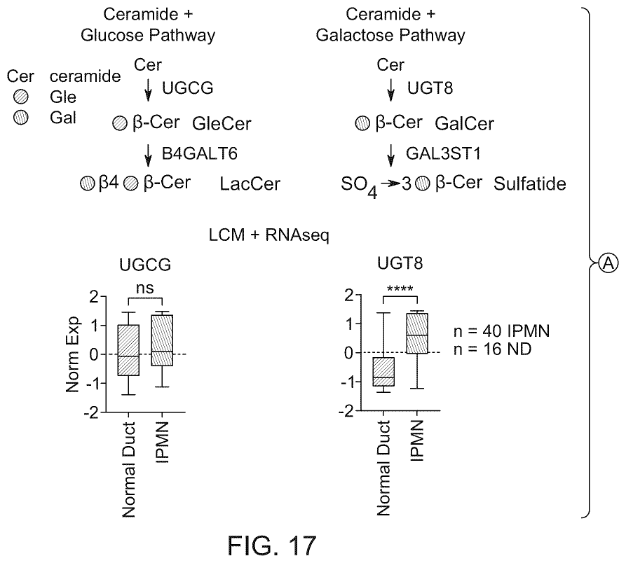

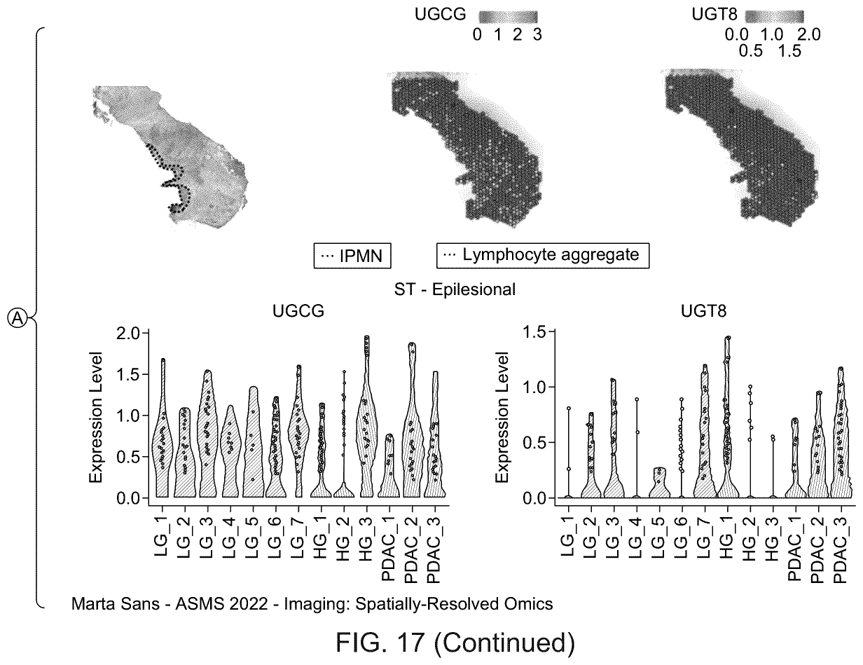

Ceramides can either get added to a compound as a glucose or a galactose. With glucose, UGCG is the enzyme responsible, which is typically further modified into LacCer, a building block for larger chain sphingolipids, such as gangliosides. On the other hand, the addition of a galactose is catalyzed by UGT8, which can then have a sulfate group added, resulting in a sulfatide. Interestingly, as shown in FIG. 17, upregulation of UGT8 expression was seen in IPMN vs normal duct, while UGCG expression was comparable. Looking at the ST data, there is a higher specificity of UGT8 expression in IPMN and PDAC areas, while UGCG was more broadly expressed in other areas of the tissue, such as stroma and lymphocyte aggregates. This data supports the upregulation of UGT8 in IPMN, which correlates with the sulfatide species observed in IPMN.

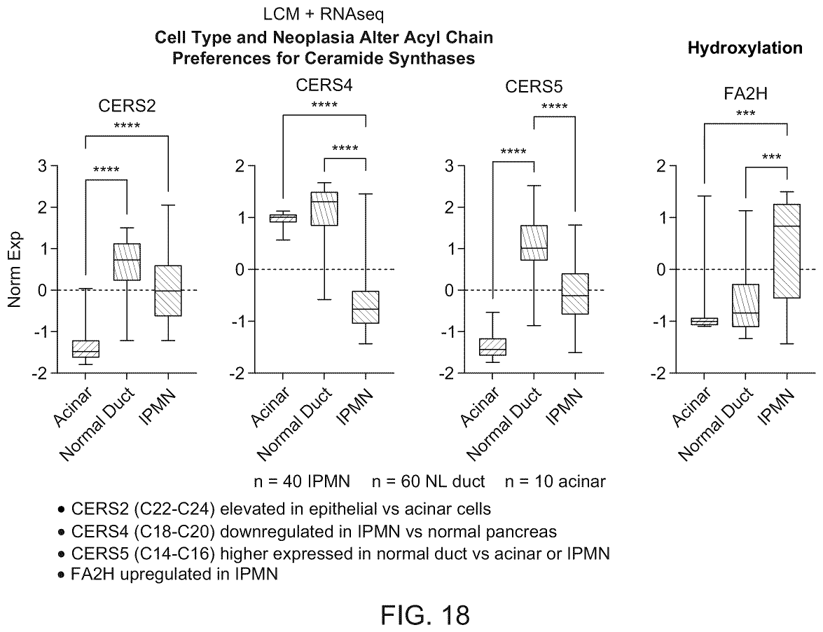

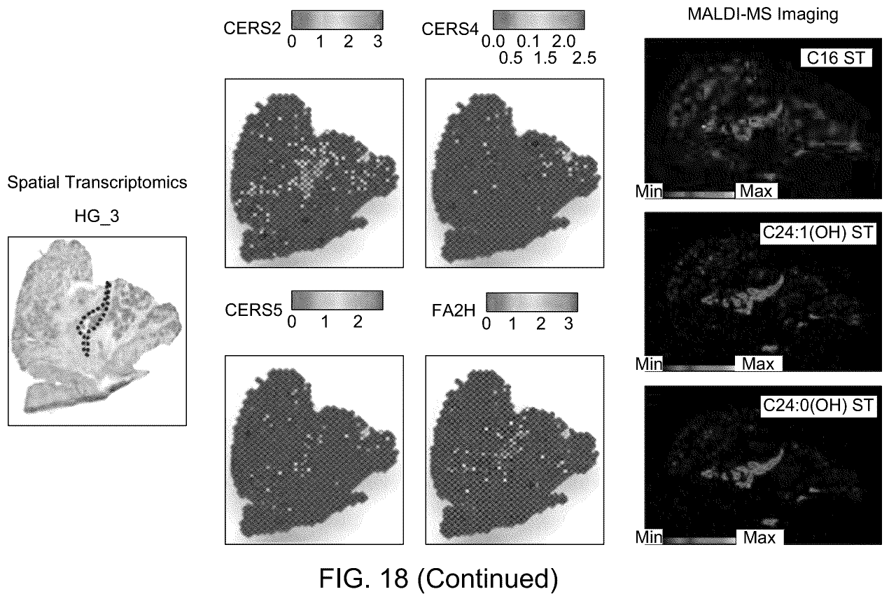

It was additionally observed that cell type and neoplasia alter the acyl chain preferences for ceramide synthases (FIG. 18). For example, CERS2 (C22-C24) was elevated in epithelial vs acinar cells; CERS4 (C18-C20) was downregulated in IPMN vs normal pancreas; CERS5 (C14-C16) showed higher expression in normal duct vs acinar or IPMN; and FA2H was upregulated in IPMN. Localization of these transcripts to the IPMN epithelium was also seen in the spatial transcriptomics dataset. Additionally, upregulation of FA2H (fatty acid 2-hydroxylase), was also seen, as well as localized the IPMN epithelium. Overall, these findings support the detection of the longer chain hydroxylated sulfatides in IPMN seen with MALDI-MS.

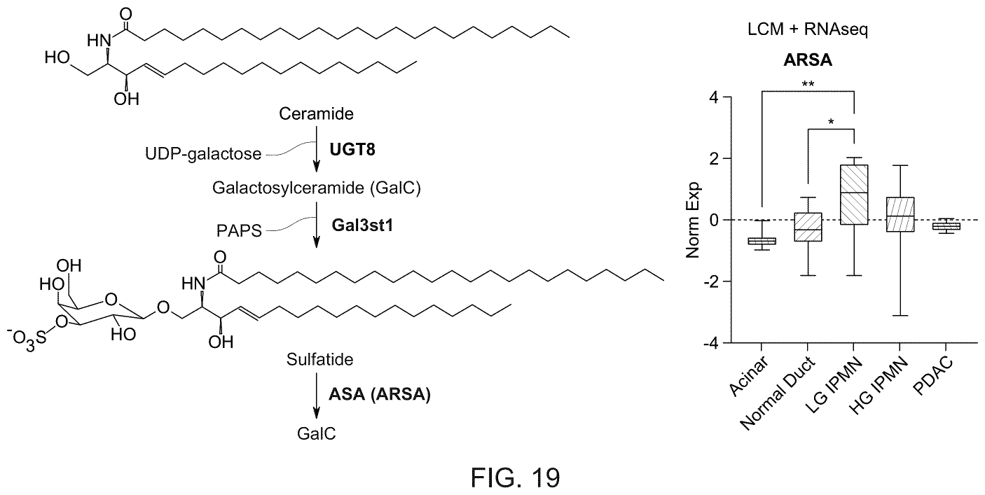

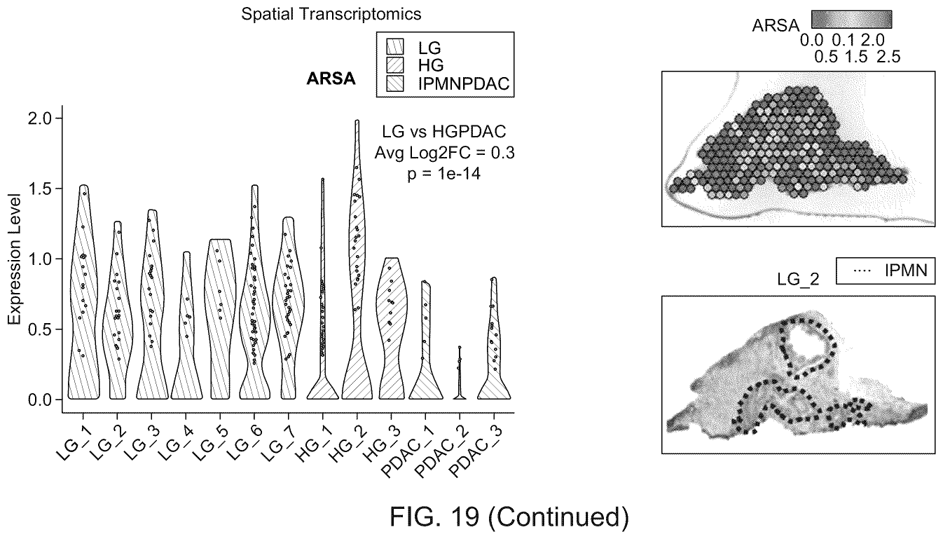

Finally, elevated arylsulfatase A levels were detected in LG-IPMN (FIG. 19). This enzyme is able to degrade sulfatide back to galactosylceramide. This was interesting, as this could be a potential compensatory mechanism in LG-IPMN to control the increased synthesis of sulfatide species, which could later be lost during progression and lead to the accumulation of sulfatide species in higher grade IPMN and cancer. This trend was also observed in the spatial transcriptomics data, higher expression in the epilesional areas in LG vs HG-PDAC, as also seen in the gene maps.

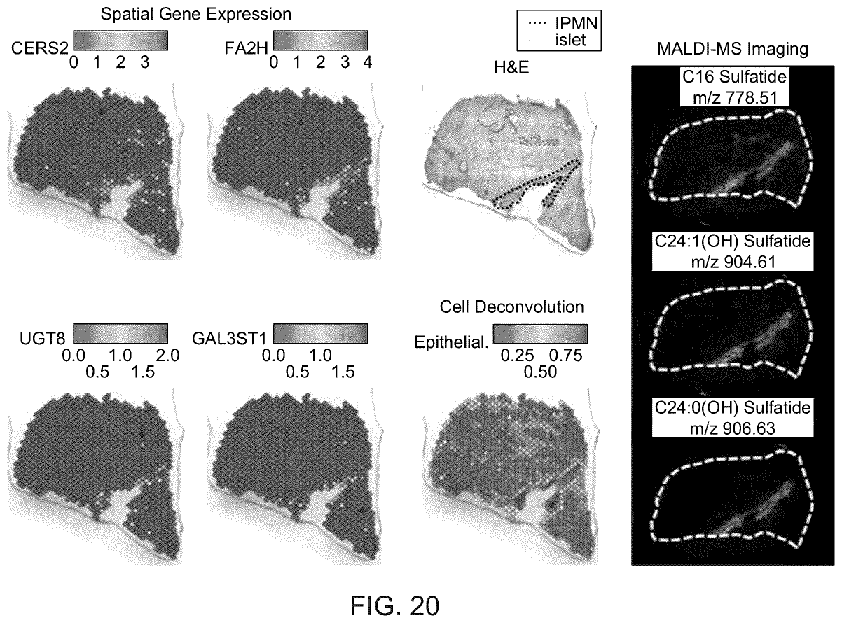

From the analyses described herein, expression of genes of interest and how they colocalize with cerebroside and sulfatide species can be evaluated. For example, FIG. 20 shows gene expression images for CERS2, FA2H, UGT8, and GAL3ST1, along with the H&E staining, and the single-cell deconvolution output highlighting the epithelial cells. When compared with the MALDI-MS imaging, the images align with each other well.

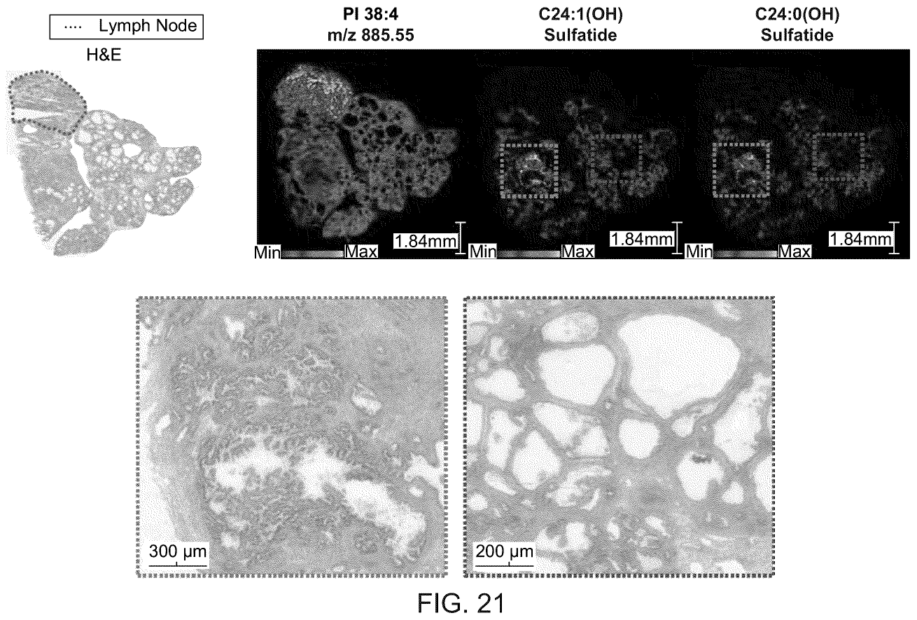

FIG. 21 shows sulfatide species detected in the murine model. Higher levels of these long-chain hydroxylated sulfatides in areas of high-grade dysplasia vs lower grade areas.

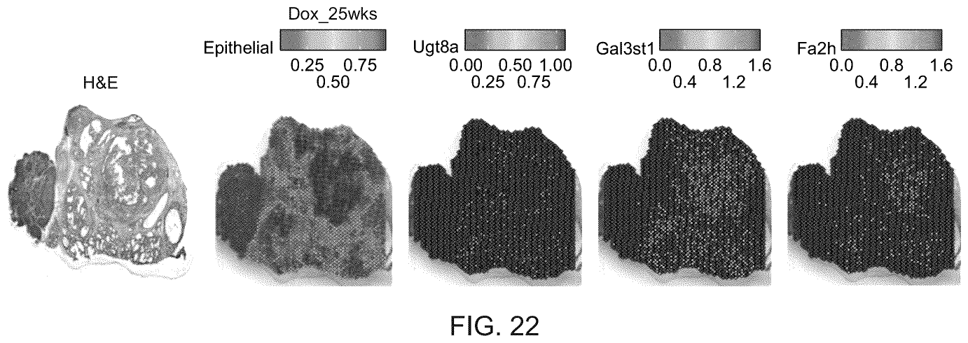

Detection of transcripts for genes that code for enzymes of interest was performed in mouse tissue (FIG. 22). In addition, localization to the epithelial compartments is shown with single-cell deconvolution, which may be useful for in vivo and functional studies.

### Example 10—Sulfatides are Increased in Cystic Fluid of Individuals Harboring Malignant IPMN

Complementary to the MALDI and spatial transcriptomic analyses of IPMN tissues described above, lipidomic profiling of cystic fluid derived from patients harboring IPMN identified several sulfatide lipid species, including the C24:1 and C24:1(OH) sulfatide, to be statistically significantly (Wilcoxon Rank Sum test 1-sided P<0.05) elevated in cystic fluid of patients with an invasive IPMN (n=7) compared to those with low-grade IPMN (n=68) (Table 1).

Levels of sulfatide species were measured via mass spectrometry in cystic fluid from patients, so measurements were all semi-quantitative values. From these levels, the area under the curve (AUC) was determined. AUC classifier performance indicates that the levels of the sulfatide species were generally higher in patients that have malignant IPMN compared to those that have low-risk ‘non-cancerous’ IPMN.

Thus, these findings provide additional supportive evidence that elevated sulfatide metabolism is associated with IPMN malignant progression. Table 4 shows area under the receiver operating characteristic curve (AUC) and corresponding 95% confidence intervals of individual sulfatides for distinguishing patients harboring a malignant IPMN (n=7) from those with a low-risk IPMN (LR; n=68). Statistical significance was based on 1-sided Wilcoxon rank sum test.

TABLE 4
Sulfatides are elevated in cystic fluid of patients with
malignant IPMN compared to those with low-grade IPMN.

| | IPMN with invasive carcinoma | |
|---|---|---|
| | vs Low Grade IPMN | |
| Lipid | AUC (95% CI) | P |
| C24(OH) sulfatide | 0.67 (0.49-0.85) | 0.08 |
| C24:1(OH) sulfatide | 0.72 (0.56-0.87) | 0.03 |
| C16 sulfatide | 0.70 (0.50-0.89) | 0.04 |
| C18 sulfatide | 0.70 (0.53-0.88) | 0.04 |
| C20 sulfatide | 0.74 (0.57-0.90) | 0.03 |
| C22 sulfatide | 0.77 (0.60-0.94) | 0.01 |
| C22:1 sulfatide | 0.76 (0.58-0.93) | 0.03 |
| C24:1 sulfatide | 0.78 (0.62-0.94) | 0.01 |

In conclusion, the present study: (1) applied a spatial multi-omics approach to investigate molecular changes associated with the development and progression of IPMN; (2) Cerebroside and sulfatide species were detected by MALDI-MS imaging with specific localization to the IPMN and PDAC epithelium; Long-chain hydroxylated sulfatides elevated in neoplasia vs normal pancreas, also elevated in the cystic fluid of patients with high risk IPMN. (3) Spatially-resolved transcriptomics with Visium and LCM+RNAseq revealed expression of gene transcripts associated with cerebroside and sulfatide metabolism localized to the IPMN and PDAC epithelium. Upregulation of UGT8 and FA2H in IPMN vs normal pancreas. ARSA levels elevated in LG-IPMN; (4) Recapitulated of the findings in a murine model of IPMN-opportunities to investigate role of cerebroside and sulfatide metabolism in vivo and in vitro.

Therefore, direct correlation of transcriptomic and lipidomic imaging provides a unique approach for molecular marker identification of non-genomic drivers in precancerous pancreatic lesions.

1. A method for determining the susceptibility of a patient having a pancreatic disorder characterized by elevated levels of long-chain hydroxylated sulfatide species progressing to pancreatic ductal adenocarcinoma (PDAC), comprising:
measuring via mass spectrometry the levels or amounts of long-chain hydroxylated sulfatide species in a biological sample obtained from the patient;
wherein increased levels or amounts of long-chain hydroxylated sulfatide species in the patient classify the patient as being susceptible to developing pancreatic ductal adenocarcinoma (PDAC) from the pancreatic disorder.

2. A method for treating or preventing progression of a pancreatic disorder to pancreatic ductal adenocarcinoma (PDAC) in a patient, comprising administering a therapeutically effective amount of an inhibitor of sulfatide metabolism to the patient.

3. A method for treating a pancreatic disorder comprising:
performing mass spectrometry imaging of sphingolipids to establish or delineate the borders of a pancreatic tumor or cyst; and
surgically resecting the tumor or cyst.

4. The method of claim 1, wherein the pancreatic disorder is characterized by elevated levels of long-chain hydroxylated sulfatide species when compared relative to healthy pancreatic tissue.

5. The method of claim 1, wherein the pancreatic disorder is selected from a pancreatic cyst or pancreatic cancer.

6. (canceled)

7. (canceled)

8. The method of claim 1, wherein the long-chain hydroxylated sulfatide species comprises a cerebroside species and/or a sulfatide species.

9. (canceled)

10. The method of claim 1, wherein the long-chain hydroxylated sulfatide species is selected from [(2R,5S,6R)-3,5-dihydroxy-2-[(E,2S,3R)-3-hydroxy-2-[[(Z)-2-hydroxytetracos-15-enoyl]amino]octadec-4-enoxy]-6-(hydroxymethyl)oxan-4-yl]hydrogen sulfate; N-(2-hydroxy-tetracosanoyl)-1-β-(3′-sulfo)-glucosyl-sphing-4-enine; [(2R,3S,6R)-3,5-dihydroxy-2-(hydroxymethyl)-6-[(E,2S,3R)-3-hydroxy-2-(tetracosanoylamino)octadec-4-enoxy]oxan-4-yl]hydrogen sulfate; [(2R,3R,4S,5S,6R)-2-[(E,2S,3R)-2-(hexadecanoylamino)-3-hydroxyoctadec-4-enoxy]-3,5-dihydroxy-6-(hydroxymethyl)oxan-4-yl]hydrogen sulfate; [(2R,3S,6R)-3,5-dihydroxy-2-(hydroxymethyl)-6-[(E,2S,3R)-3-hydroxy-2-(octadecanoylamino)octadec-4-enoxy]oxan-4-yl]hydrogen sulfate; [(2R,5S,6R)-3,5-dihydroxy-2-[(E,2S,3R)-3-hydroxy-2-(icosanoylamino)octadec-4-enoxy]-6-(hydroxymethyl)oxan-4-yl]hydrogen sulfate; or [(2R,5S,6R)-2-[(E,2S,3R)-2-(docosanoylamino)-3-hydroxyoctadec-4-enoxy]-3,5-dihydroxy-6-(hydroxymethyl)oxan-4-yl]hydrogen sulfate.

11. The method of claim 8, wherein the sulfatide species is selected from C24(OH) sulfatide, C24:1(OH) sulfatide, C16 sulfatide, C18 sulfatide, C20 sulfatide, C22 sulfatide, C22:1 sulfatide, and C24:1 sulfatide.

12. The method of claim 8, wherein the cerebroside species has a glucose sugar and is a glucocerebroside or a glucosylceramide, or wherein the cerebroside species has a galactose sugar and is a galactocerebroside or galactosylceramide.

13. The method of claim 2, wherein the inhibitor of sulfatide metabolism is administered prior to development of pancreatic ductal adenocarcinoma (PDAC) from the pancreatic disorder.

14. The method of claim 1, wherein the patient having the pancreatic disorder has pre-neoplastic lesions.

15. (canceled)

16. (canceled)

17. The method of claim 1, wherein the pancreatic disorder comprises a point mutation in the KRAS oncogene.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the pancreatic disorder comprises low-grade or high-grade epithelial dysplasia.

30. The method of claim 3, wherein the treatment further comprises a surgical procedure, a chemotherapeutic treatment, or a radiation treatment.

31. The method of claim 30, wherein the surgical procedure is selected from partial or complete surgical removal of cancerous tissue.

32. The method of claim 31, wherein the partial or complete surgical procedure comprises a Whipple procedure, distal pancreatectomy, or total pancreatectomy.

33. The method of claim 30, wherein the chemotherapeutic treatment comprises administration of one or more chemotherapeutic drugs or pharmacological substances.

34. The method of claim 30, wherein the radiation treatment comprises administering a conventional or standard fraction radiation therapy stereotactic body radiation (SBRT) to the affected tissue.

35. (canceled)

36. The method of claim 33, wherein the one or more pharmacological substances is selected from an immunotherapy drug.

37. (canceled)

38. (canceled)

39. (canceled)

40. The method of claim 1, wherein the mass spectrometry comprises MALDI-MS imaging.

41. (canceled)
